(19) United States
(12) Patent Application Publication
(10) Pub. No.: US 2021/0057046 A1
(43) Pub. Date: Feb. 25, 2021

(54) METHODS AND SYSTEMS FOR ANALYZING MICROBIOTA

(71) Applicant: Freenome Holdings, Inc., South San Francisco, CA (US)

(72) Inventors: Yaping LIU, South San Francisco, CA (US); Daniel DELUBAC, South San Francisco, CA (US); Imran S. HAQUE, South San Francisco, CA (US)

(21) Appl. No.: 17/035,278

(22) Filed: Sep. 28, 2020

Related U.S. Application Data
(63) Continuation of application No. PCT/US2019/024942, filed on Mar. 29, 2019.
(60) Provisional application No. 62/650,156, filed on Mar. 29, 2018.

(51) Int. Cl. G16B 30/10; G16B 20/00; G16B 40/30

(52) U.S. Cl. CPC G16B 30/10; G16B 40/30; G16B 20/00

(57) ABSTRACT

Systems, media, methods, and kits disclosed herein can be used to analyze human microbiota for the detection of a condition (e.g., a disease or condition). Further, the systems, media, methods, and kits disclosed herein can utilize machine learning algorithms to analyze samples with high accuracy. In an aspect, a classifier capable of distinguishing a population of subjects based on microbiome composition may comprise: a plurality of microbiome-associated features associated with two or more classes of subjects inputted into a machine learning model, wherein the features comprise the microbiome species and abundance of microbiome elements, wherein the features are derived from a taxonomic community composition analysis of a cell-free nucleic acid sample in a population of subjects; wherein the features contribute to a classifier sensitivity of greater than 50% and a classifier specificity of greater than 85% to distinguish the population of subjects into two or more classes.

### CROSS REFERENCE TO RELATED APPLICATIONS

This application claims the benefit of U.S. provisional patent application 62/650,156, filed Mar. 29, 2018, the contents of which are hereby incorporated in its entirety.

### BACKGROUND OF THE INVENTION

Human microbiota is a complex and dynamic ensemble of microorganisms that resides in the human body. The human gut microbiota contains hundreds of trillions of microorganisms, including more than 1,000 different known species of bacteria. These bacteria harbor more than 3 million genes, which is more than 100 times larger than the human genome. Approximately one-third of the gut microbiota is common to most people, while two-thirds are specific to each individual. Thus, an individual's microbiota can provide information on variations between individuals including, for example, information on diseases or conditions such as cancer.

Colorectal adenomas are considered precursor lesions of most cases of colorectal carcinoma. Advanced adenoma can be defined as a subset of adenoma in which the lesion size measures 10 mm or more and contains a substantially villous component or high-grade dysplasia. Only about 1-10% of people with adenomas develop colorectal carcinoma, while significantly more advanced adenoma patients eventually advance to colorectal carcinoma. For example, projections of 10-year cumulative risk for advanced adenoma progressing to colorectal cancer increase from 25.4% at age 55 years to 42.9% at age 80 years in women, and from 25.2% at age 55 years to 39.7% at age 80 years in men. Early detection and removal of advanced adenomas can dramatically decrease the incidence of colorectal carcinoma.

As with many other complex diseases, the susceptibility and progression of cancer are primarily influenced by gene-environment interactions. Tremendous progress has been made to explore the genetics and the molecular mechanisms that underlie carcinogenesis. The understanding of environmental factors that influence cancer susceptibility and progression, however, is still very limited. The microbiota is composed of bacteria, archaea, eukaryotes, and viruses that reside in different sites of the human body, including the gut and circulating blood. The microbiota is an example of an environmental factor that can influence carcinogenesis.

Current methods of using gut microbiota as disease indicators can be costly and invasive. For example, total colonoscopy is the current gold standard for screening advanced adenoma and colorectal carcinoma. However, due to its high cost and invasiveness, total colonoscopies may have limited application for early stage whole population screening. Alternative non-invasive screening methods include fecal occult blood test (FOBT), the fecal immunochemical test (FIT), the fecal-based DNA test, and the blood-based DNA test (the SEPT9 assay). However, these methods may struggle to detect advanced adenoma, a significant precursor to colorectal cancer. What is therefore needed are approaches to harness the informative power of gut microbiota in a non-invasive manner to detect advanced adenoma, colorectal cancer and distinguish different stages. What is also needed are methods of using microbiota to provide information to stratify individuals based on other diseases or conditions, and treatment responsiveness.

### SUMMARY OF THE INVENTION

Provided herein are systems and methods for identifying microbiome communities in an individual and for identifying a disease or condition in an individual. By using cell-free nucleic acids, these approaches are non-invasive and therefore advantageous over invasive techniques used to sample microbiota. The present disclosure provides a method that utilizes sequencing data from an individual and reference genomic sequences to determine which sequence information is from the individual's own genome and which sequence information is microbiome-derived. In particular, sequence information can be compared to a human reference sequence to detect which sequences are human and to separate these sequences to focus on characterizing and analyzing non-human sequences in the sample. Machine learning analysis methods are used to identify classifiers to stratify populations of individuals based on disease state or treatment responsiveness.

In a first aspect, the disclosure provides a classifier capable of distinguishing a population of individuals based on microbiome composition, comprising: a plurality of microbiome-associated features associated with two or more classes of individuals inputted into a machine learning model; wherein the features comprise the microbiome species and abundance of microbiome elements; wherein the microbiome-associated features are derived from a taxonomic community composition analysis of a cfDNA sample in a population of individuals; wherein the microbiome-associated features contribute to a classifier sensitivity of greater than 50%; and wherein the microbiome-associated features contribute to a classifier specificity of greater than 85% to distinguish the population of individuals into two or more classes.

In one embodiment, the classifier is constructed according to one or more of: linear discriminant analysis (LDA); partial least squares (PLS); random forest; k-nearest neighbor (KNN); support vector machine (SVM) with radial basis function kernel (SVMRadial); SVM with linear basis function kernel (SVMLinear); and SVM with polynomial basis function kernel (SVMPoly).

In one embodiment, the population of individuals contains one or more individuals having advanced adenoma and/or colorectal cancer, and the classifier is capable of distinguishing individuals with advanced adenoma and colorectal cancer from the total population of individuals based on the plurality of microbiome-associated features.

In one embodiment, the classifier is capable of differentiating between microbiomes associated with advanced adenoma and colorectal cancer based on the plurality of microbiome-associated features.

In one embodiment, the microbiome composition features are associated with a set of taxa comprising at least one of: Alistipes (genus), Barnesiella (genus), Bifidobacterium (genus), Clostridium (genus), Lactobacillus (genus), Odoribacter (genus), Prevotella (genus), Flavonifractor (genus), Roseburia (genus), Ruminococcus (genus), Veillonella (genus), Akkermansia (genus), Bacteroides (genus), Pseudobutyrivibrio (genus), Collinsella (genus), Coprococcus (genus), Desulfovibrionales (order), Dialister (genus), Faecalibacterium (genus), and Streptococcus (genus).

In one embodiment, the microbiome composition features are associated with a set of taxa comprising at least one of: Clostridiaceae (family), Prevotellaceae (family), Oscillospiraceae (family), Gammaproteobacteria (class), Proteobacteria (phylum), Eggerthella (genus), Anaerosporobacter (genus), Erysipelothrix (genus), Legionella (genus), Parabacteroides (genus), Barnesiella (genus), Actinobacillus (genus), Haemophilus (genus), Megasphaera (genus), Marvinbryantia (genus), Butyricicoccus (genus), Bilophila (genus), Oscillibacter (genus), Butyricimonas (genus), Sarcina (genus), Pectobacterium (genus), Eubacterium (genus), Subdoligranulum (genus), Cronobacter (genus), Lachnospira (genus), Blautia (genus), Peptostreptococcaceae (family), Veillonellaceae (family), Erysipelotrichaceae (family), Christensenellaceae (family), Erysipelotrichales (order), Erysipelotrichia (class), Actinobacillus porcinus (species), Pasteurellaceae (family), Pasteurellales (order), Flavonifractor plautii (species), Lactobacillales (order), Lachnospiraceae bacterium 2_1_58FAA (species), Bacilli (class), bacterium NLAE-z1-P430 (species), Parasutterella (genus), Parasutterella excrementihominis (species), Coriobacteriaceae (family), uncultured Coriobacteriia bacterium (species), Coriobacteriales (order), Bacteroides fragilis (species), Holdemania (genus), Porphyromonadaceae (family), Chlamydiae/Verrucomicrobia group (superphylum), Eggerthella lenta (species), Verrucomicrobia (phylum), Bacteroidales (order), Bacteroidia (class), Bacteroidetes (phylum), Bacteroidetes/Chlorobi group (superphylum), Verrucomicrobiae (class), Verrucomicrobiales (order), Verrucomicrobiaceae (family), Dorea (genus), Deltaproteobacteria (class), delta/epsilon subdivisions (subphylum), Bacillales incertae sedis (no rank), Desulfovibrionales (order), Eubacteriaceae (family), Acidaminococcaceae (family), Rhodospirillales (order), Rhodospirillaceae (family), Bacillales (order), Alistipes putredinis (species), Bacillaceae (family), Selenomonadales (order), Gammaproteobacteria (class), Negativicutes (class), bacterium NLAE-z1-P562 (species), Enterobacteriales (order), Enterobacteriaceae (family), Streptococcaceae (family), Cronobacter sakazakii (species), Streptococcus (genus), Burkholderiales (order), Betaproteobacteria (class), Sutterellaceae (family), Ruminococcaceae (family), butyrate-producing bacterium SR1/1 (species), Sphingobacteriales (order), Bacillales Family XI. Incertae Sedis, Oceanospirillales (order), Finegoldia (genus), Rikenellaceae (family), Bilophila wadsworthia (species), Clostridiales (order), Clostridia (class), Clostridium lavalense (species), Odoribacter splanchnicus (species), organismal metagenomes (no rank), Anaerostipes (genus), Actinobacteria (class), bacterium NLAE-z1-H54 (species), Actinobacteridae spp. (no rank), Roseburia sp. 11SE38 (species), Bifidobacteriaceae (family), Bifidobacteriales (order), Finegoldia magna (species), Finegoldia (genus), and Peptoniphilus (genus).

In a second aspect, the disclosure provides a method of classifying an individual microbiome in a cell-free nucleic acid (cfNA) sample to identify a disease or condition of a subject comprising: (a) mapping a plurality of sequence reads obtained from sequencing a cell-free nucleic acid sample to a reference nucleic acid sequence; (b) separating sequence reads that do not map to a reference nucleic acid sequence, thereby providing presumed microbiome sequence reads; (c) comparing the presumed microbiome sequence reads to a reference microbiome nucleic acid sequence, wherein the presumed microbiome sequence reads that map to the reference microbiome nucleic acid sequence are actual microbiome sequence reads; and (d) applying a predictive model for classifying the subject to a disease or condition associated with the actual microbiome sequence reads of the subject.

In one embodiment, the applying a predictive model comprises using a computer readable medium, wherein the computer readable medium comprises a plurality of microbiome features and a classifier, wherein each microbiome feature of the plurality of microbiome features maps the microbiome information to a respective value, the classifier capable of distinguishing at least two groups based on the plurality of microbiome features.

In one embodiment, the cell-free nucleic acid sample is: blood, urine, saliva, sweat, or a fraction thereof.

In one embodiment, the cell-free nucleic acid sample comprises serum, plasma, a buffy coat layer, erythrocytes, platelets, or exosomes.

In one embodiment, the plasma is platelet-rich plasma.

In one embodiment, the cell-free nucleic acid sample is free offecal matter.

In one embodiment, the reference nucleic acid sequence is a human reference genome.

In one embodiment, the human reference genome is GrCH38, GrCH37, NA12878, or GM12878.

In one embodiment, the sequences are mapped to species of microbiota selected from Clostridiaceae (family), Prevotellaceae (family), Oscillospiraceae (family), Gammaproteobacteria (class), Proteobacteria (phylum), Eggerthella (genus), Anaerosporobacter (genus), Erysipelothrix (genus), Legionella (genus), Parabacteroides (genus), Barnesiella (genus), Actinobacillus (genus), Haemophilus (genus), Megasphaera (genus), Marvinbryantia (genus), Butyricicoccus (genus), Bilophila (genus), Oscillibacter (genus), Butyricimonas (genus), Sarcina (genus), Pectobacterium (genus), Eubacterium (genus), Subdoligranulum (genus), Cronobacter (genus), Lachnospira (genus), Blautia (genus), Peptostreptococcaceae (family), Veillonellaceae (family), Erysipelotrichaceae (family), Christensenellaceae (family), Erysipelotrichales (order), Erysipelotrichia (class), Actinobacillus porcinus (species), Pasteurellaceae (family), Pasteurellales (order), Flavonifractor plautii (species), Lactobacillales (order), Lachnospiraceae bacterium 2_1_58FAA (species), Bacilli (class), bacterium NLAE-z1-P430 (species), Parasutterella (genus), Parasutterella excrementihominis (species), Coriobacteriaceae (family), uncultured Coriobacteriia bacterium (species), Coriobacteriales (order), Bacteroides fragilis (species), Holdemania (genus), Porphyromonadaceae (family), Chlamydiae/Verrucomicrobia group (superphylum), Eggerthella lenta (species), Verrucomicrobia (phylum), Bacteroidales (order), Bacteroidia (class), Bacteroidetes (phylum), Bacteroidetes/Chlorobi group (superphylum), Verrucomicrobiae (class), Verrucomicrobiales (order), Verrucomicrobiaceae (family), Dorea (genus), Deltaproteobacteria (class), delta/epsilon subdivisions (subphylum), Bacillales incertae sedis (no rank), Desulfovibrionales (order), Eubacteriaceae (family), Acidaminococcaceae (family), Rhodospirillales (order), Rhodospirillaceae (family), Bacillales (order), Alistipes putredinis (species), Bacillaceae (family), Selenomonadales (order), Gammaproteobacteria (class), Negativicutes (class), bacterium NLAE-z1-P562 (species), Enterobacteriales (order), Enterobacteriaceae (family), Streptococcaceae (family), Cronobacter sakazakii (species), Streptococcus (genus), Burkholderiales (order), Betaproteobacteria (class), Sutterellaceae (family), Ruminococcaceae (family), butyrate-producing bacterium SR1/1 (species), Sphingobacteriales (order), Bacillales Family XI. Incertae Sedis, Oceanospirillales (order), Finegoldia (genus), Rikenellaceae (family), Bilophila wadsworthia (species), Clostridiales (order), Clostridia (class), Clostridium lavalense (species), Odoribacter splanchnicus (species), organismal metagenomes (no rank), Anaerostipes (genus), Actinobacteria (class), bacterium NLAE-z1-H54 (species), Actinobacteridae spp. (no rank), Roseburia sp. 11SE38 (species), Bifidobacteriaceae (family), Bifidobacteriales (order), Finegoldia magna (species), Finegoldia (genus), and Peptoniphilus (genus).

In one embodiment, the sequences are mapped to species of microbiota selected from Propionibacterium spp., Candidatus Zinderia spp., Dasheen mosaic virus, Vicia cryptic virus, Comamonas spp., Caulobacter spp., Acinetobacter spp., Burkholdreia spp., Micrococcus spp., Candidatus Sulcia spp., Torque teno virus, Polaromonas spp., Pseudomonas spp., Acinetobacter spp., Cupriavidus spp., Dietzia spp., Neisseria spp., Propionibacterium spp., Stenotrophomonas spp., and combinations thereof.

In one embodiment, the sequences are mapped to species of microbiota selected from Propionibacterium acnes, Candidatus Zinderia insecticola, Dasheen mosaic virus, Vicia cryptic virus, Comamonas spp., Caulobacter spp., Acinetobacter spp., Burkholdreia spp., Micrococcus luteus, Candidatus Sulcia muelleri, Torque teno virus, Polaromonas spp., Pseudomonas spp., Acinetobacter johnsonii, Cupriavidus spp., Dietzia spp., Neisseria spp., Propionibacterium granulosum, Stenotrophomonas maltophilia, and combinations thereof.

In one embodiment, the sequences are mapped to species of microbiota selected from Propionibacterium acnes, Candidatus Zinderia insecticola, Dasheen mosaic virus, Vicia cryptic virus, and combinations thereof.

In one embodiment, the sequences are mapped to species of microbiota selected from Propionibacterium acnes, Candidatus Zinderia insecticola, Dasheen mosaic virus, Vicia cryptic virus, Comamonas spp., Caulobacter spp., Acinetobacter spp., and combinations thereof.

In one embodiment, the sequences are mapped to species of microbiota selected from Propionibacterium acnes, Candidatus Zinderia insecticola, Dasheen mosaic virus, Vicia cryptic virus, Comamonas spp., Caulobacter spp., Acinetobacter spp., Burkholdreia spp., Micrococcus luteus, Candidatus Sulcia muelleri, Torque teno virus, and combinations thereof.

In one embodiment, the method further comprises generating a feature matrix from the actual microbiome sequence reads.

In various embodiments, the microbiome features are selected from microbiota species, relative abundance of microbiota species, age of subject, sex of subject, disease stage, high or low fiber content in diet, and treatment responder or non-responder.

In one embodiment, the actual microbiome sequence reads are used to determine the relative abundance of microbiota.

In one embodiment, the relative abundance of microbiota is a relative abundance of a plurality of species of microbiota.

In one embodiment, the method further comprises performing a principal component analysis of the feature matrix.

In one embodiment, the method further comprises applying machine learning to the principal component analysis.

In one embodiment, the machine learning comprises a random forest, gradient boost tree, logistic regression, neural network, or a combination thereof.

In one embodiment, the disease is inflammatory bowel disease.

In one embodiment, the disease is cancer.

In one embodiment, the cancer is advanced adenoma.

In one embodiment, the cancer is colorectal cancer.

In one embodiment, the actual microbiome sequence reads identify the disease or condition of the subject at a sensitivity of 40% or greater and a specificity of 70% or greater.

In one embodiment, the sensitivity is 50% or greater and the specificity is 80% or greater.

In one embodiment, the sequencing is selected from: whole genome sequencing, whole exome sequencing, and targeted sequencing.

In one embodiment, the sample is processed through plasma isolation, cfDNA extraction, sequencing library preparation, and deep whole genome sequencing (WGS).

In one embodiment, the comparing of the presumed microbiome sequence reads comprises mapping taxonomic microbiota community composition of the cfNA sample using Metagenomic Phylogenetic Analysis to generate a relative abundance score of microbiota represented in the cfNA sample.

In a third aspect, the disclosure provides a method for classifying an advanced adenoma or colorectal cancer, comprising: (a) assaying a biological sample from a subject by sequencing, array hybridization, or nucleic acid amplification to determine sequences of gene expression products in the biological sample, wherein the gene expression products are associated with an advanced adenoma or colorectal cancer condition; (b) mapping sequences of the gene expression products to microbiota, (c) classifying the biological sample as positive or negative for the advanced adenoma or colorectal cancer using a trained algorithm to process the mapped sequences, wherein the trained algorithm classifies biological samples as negative for the advanced adenoma or colorectal cancer at an accuracy of at least 90%; and (d) outputting a report on a computer screen that is indicative of the classification of the biological sample as positive or negative for the advanced adenoma or colorectal cancer.

In one embodiment, the sequences are inputted into a machine learning algorithm to create a classifier capable of classifying the biological sample.

In one embodiment, the classifying the biological sample is performed by a classifier trained and tested using a statistical method selected from the group consisting of support vector machines (SVM), linear discriminant analysis (LDA), k-nearest neighbor analysis (KNN), and random forest (RF).

In a fourth aspect, the disclosure provides a method of diagnosing advanced adenoma or colorectal cancer, comprising: (a) obtaining a biological sample comprising cfDNA from a subject; (b) assaying by sequencing, array hybridization, or nucleic acid amplification gene expression products of the biological sample, which gene expression products are associated with an advanced adenoma or colorectal cancer; (c) comparing to an amount in a control sample, an amount of one or more gene expression products in the biological sample to determine one or more differential gene expression product levels between the biological sample and the control sample; (d) classifying the biological sample by inputting the one or more differential gene expression product levels into a trained algorithm, and (e) outputting a report on a computer screen that identifies the biological sample as negative for the advanced adenoma or colorectal cancer if the trained algorithm classifies the biological sample as negative for the advanced adenoma or colorectal cancer at a specified confidence level.

In one embodiment, the trained algorithm classifies biological samples as negative for advanced adenoma or colorectal cancer at an accuracy of at least 90%, wherein a plurality of technical factor variables is removed from data comprising the amounts of the one or more gene expression products based on one or more of the differential gene expression product levels and normalized prior to or during classification, wherein the plurality of technical factor variables is selected from the group consisting of a collection source, a collection method, a collection media, a RNA integrity number, a whole transcriptome amplification yield, a sense strand yield, a hybridization site, a hybridization quality, and an experiment batch.

In one embodiment, the classifying the biological sample is performed by a classifier that is trained and tested by a statistical method selected from the group consisting of support vector machines (SVM), linear discriminant analysis (LDA), k-nearest neighbor analysis (KNN), and random forest (RF).

In a fifth aspect, the disclosure provides a method of detecting presence of cancer in an individual comprising: (a) mapping a plurality of sequence reads obtained from sequencing a cell-free nucleic acid sample to a reference nucleic acid sequence; (b) separating sequence reads that do not map to the reference nucleic acid sequence, thereby providing presumed microbiome sequence reads; (c) comparing the presumed microbiome sequence reads to a reference microbiome nucleic acid sequence, wherein the presumed microbiome sequence reads that map to the reference microbiome nucleic acid sequence are actual microbiome sequence reads; and (d) applying a predictive model to the actual microbiome sequence reads to classify the subject to detect the presence of cancer in the subject.

In a sixth aspect, the disclosure provides a system for classifying subjects based on microbiome composition comprising: (a) a computer readable medium comprising the classifier; and (b) one or more processors for executing instructions stored on the computer readable medium.

In one embodiment, the system comprises a classification circuit that is configured as a machine learning classifier selected from a linear discriminant analysis (LDA) classifier, a quadratic discriminant analysis (QDA) classifier, a support vector machine (SVM) classifier, a random forest (RF) classifier, a linear kernel support vector machine classifier, a first or second order polynomial kernel support vector machine classifier, a ridge regression classifier, an elastic net algorithm classifier, a sequential minimal optimization algorithm classifier, a naive Bayes algorithm classifier, and a NMF predictor algorithm classifier.

In one embodiment, the system comprises means for performing any of the preceding methods.

In one embodiment, the system comprises one or more processors configured to perform any of the preceding methods.

In one embodiment, the system comprises modules that respectively perform the steps of any of the preceding methods.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent that publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a computer system that is programmed or otherwise configured to implement methods provided herein.

FIG. 2 shows a principal component analysis (PCA) plot of reads mapped to all human microbiome reference genome showing distinct separation of advanced adenoma samples from healthy samples and inflammatory bowel disease samples.

FIG. 3 shows a receiver operating characteristic (ROC) curve for distinguishing advanced adenoma samples and healthy samples based on normalized number of reads mapped to the human microbiome genome.

FIG. 4 shows a graph of a feature importance rank plot for the classification of samples from advanced adenoma (AA) vs. healthy individuals. Microbial elements represented in the sequences are shown as a measure of relative feature importance.

### DETAILED DESCRIPTION OF THE INVENTION

While various embodiments of the invention have been shown and described herein, it will be obvious to those having ordinary skill in the art that such embodiments are provided by way of example only. Numerous variations, changes, and substitutions can occur to those having ordinary skill in the art without departing from the invention. It should be understood that various alternatives to the embodiments of the invention described herein can be employed.

### Definitions

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject” refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. The subject may be displaying a symptom(s) indicative of a health or physiological state or condition of the subject, such as a disease or disorder of the subject. As an alternative, the subject can be asymptomatic with respect to such health or physiological state or condition.

As used herein, the term “sample” generally refers to a biological sample obtained from or derived from one or more subjects. Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck), or a cell-free DNA collection tube (e.g., Streck). Cell-free biological samples may be derived from whole blood samples by fractionation.

The term “nucleic acid” used herein refers to a polynucleotide comprising two or more nucleotides, i.e., a polymeric form of nucleotides of any length, either deoxyribonucleotides (dNTPs) or ribonucleotides (rNTPs), or analogs thereof. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent. A “variant” nucleic acid is a polynucleotide having a nucleotide sequence identical to that of its original nucleic acid except having at least one nucleotide modified, for example, deleted, inserted, or replaced, respectively. The variant may have a nucleotide sequence at least about 80%, 90%, 95%, or 99%, identity to the nucleotide sequence of the original nucleic acid.

The term “circulating free DNA” or “cell-free DNA” (cfDNA) refers to DNA found in circulation of a subject. Studies reveal that much of the circulating nucleic acids in blood arise from necrotic or apoptotic cells and greatly elevated levels of nucleic acids from apoptosis is observed in diseases such as cancer. Particularly for cancer, where the circulating DNA bears hallmark signs of the disease including mutations in oncogenes, microsatellite alterations, and, for certain cancers, viral genomic sequences, DNA or RNA in plasma has become increasingly studied as a potential biomarker for disease. For example, a quantitative assay for low levels of circulating tumor DNA in total circulating DNA could serve as a better marker for detecting the relapse of colorectal cancer compared with carcinoembryonic antigen, the standard biomarker used clinically. Genotyping of circulating cells in plasma to detect activating mutations in epidermal growth factor receptors in cancer patients could affect drug treatment. Thus, circulating DNA in plasma is a useful species in cancer detection and treatment. Circulating DNA has also been useful in healthy patients for fetal diagnostics. For example, fetal DNA circulating in maternal blood could serve as a marker for gender, rhesus D status, fetal aneuploidy, and sex-linked disorders. A strategy for detecting fetal aneuploidy by shotgun sequencing of cell-free DNA taken from a maternal blood sample can replace more invasive and risky techniques such as amniocentesis or chorionic villus sampling. The term “cell-free fraction” of a biological sample used herein refers to a fraction of the biological sample that is substantially free of cells. For example, the cell-free fraction of a blood sample may be blood serum or blood plasma. The term “substantially free of cells” used herein refers to a preparation from the biological sample comprising fewer than about 20,000 cells per mL, preferably fewer than about 2,000 cells per mL, more preferably fewer than about 200 cells per mL, most preferably fewer than about 20 cells per mL. In contrast to some methods, genomic DNA may not be excluded from the acellular sample, and typically comprises from about 50% to about 90% of the nucleic acids that are present in the sample.

In this disclosure the terms “colorectal cancer (CRC)” and “colon cancer” have the same meaning and refer to a cancer of the large intestine (colon), the lower part of the human digestive system, although rectal cancer often more specifically refers to a cancer of the last several inches of the colon, the rectum. A “colorectal cancer cell” is a colon epithelial cell possessing characteristics of colon cancer and encompasses a precancerous cell, which is in the early stages of conversion to a cancer cell or which is predisposed for conversion to a cancer cell. Such cells may exhibit one or more phenotypic traits characteristic of the cancerous cells.

The term “derived from” used herein refers to an origin or source, and may include naturally occurring, recombinant, unpurified, or purified molecules. A nucleic acid derived from an original nucleic acid may comprise the original nucleic acid, in part or in whole, and may be a fragment or variant of the original nucleic acid. A nucleic acid derived from a biological sample may be purified from that sample.

As used herein, the term “diagnose” or “diagnosis” of a status or outcome includes predicting or diagnosing the status or outcome, determining predisposition to a status or outcome, monitoring treatment of patient, diagnosing a therapeutic response of a patient, and prognosis of status or outcome, progression, and response to particular treatment.

As used herein, the term “microbiota” refers to the set of microorganisms present within a subject, an individual, usually an individual mammal and more usually a human individual. The microbiota may include pathogenic species; species that constitute the normal flora of one tissue, e.g., skin and oral cavity, but are undesirable in other tissues, e.g., blood and lungs; and commensal organisms found in the absence of disease. A subset of the microbiome is the virome, which comprises the viral components of the microbiome.

The term “microbiome component” as used herein refers to an individual strains or species, The component may be a viral component, a bacterial component, or a fungal component.

A “target nucleic acid” as used herein refers to a nucleic acid, DNA or RNA, to be detected. A target nucleic acid derived from an organism is a polynucleotide that has a sequence derived from that of the organism and is specific to the organism. A target nucleic acid derived from a pathogen refers to a polynucleotide having a polynucleotide sequence derived from that specific the pathogen.

Provided herein are systems and methods for identifying microbiome communities in a subject and for identifying a disease or condition in a subject. By using cell-free nucleic acids, these approaches may be non-invasive or minimally invasive and therefore advantageous over invasive techniques used to sample microbiota. The present disclosure provides a method that utilizes sequencing data from an individual and reference genomic sequences to determine which sequence information is from the individual's own genome and which sequence information is microbiome-derived. In particular, sequence information can be compared to a human reference sequence to detect which sequences are human. The remaining sequences, therefore, are presumed to be non-human and can comprise sequences from microbiota. These non-human sequences can then be compared to other reference sequences such as bacterial sequences. Exemplary bacterial sequences can be obtained, for example, from the Human Microbiome Project. By performing this process in samples from healthy subjects and subjects with a particular disease or condition, and comparing the differences in microbiome sequences, signatures for the disease or condition can be determined.

The present disclosure provides systems and methods for analyzing human microbiota, for example, by analyzing cell-free nucleic acids derived from human microbiota to detect a disease or condition, for example, advanced adenoma, colorectal carcinoma, and inflammatory bowel disease.

Current methods of using gut microbiota as disease indicators can be costly and invasive. For example, total colonoscopy is the current gold standard for screening advanced adenoma and colorectal carcinoma. However, due to its high cost and invasiveness, total colonoscopies may have limited application for early stage whole population screening. Alternative non-invasive screening methods include fecal occult blood test (FOBT), the fecal immunochemical test (FIT), the fecal-based DNA test, and the blood-based DNA test (the SEPT9 assay). However, these methods may struggle to detect advanced adenoma, a significant precursor to colorectal cancer, as evidenced by the sensitive and specificity percentages shown in TABLE 1.

TABLE 1
Sensitivity and specificity of CRC screening methods

| | FIT | Fecal DNA | SEPT9 |
|---|---|---|---|
| Sensitivity | 24% | 42% | 18% |
| Specificity | 94% | 87% | 80% |

The present disclosure provides non-invasive systems and methods for detecting gut microbiota with increased sensitivity and specificity, while simultaneously lowering the cost as compared to traditional methods. In particular, the present disclosure provides systems and methods for detecting communities of microbiota and for diagnosing diseases such as cancer.

As a part of the tumor microenvironment, the gastrointestinal microbiome participates in the development of gastrointestinal tract malignancies. Namely, the dysbiosis of gut microbiota has been linked to the development of colorectal adenocarcinoma. Certain species of gut microbes can induce inflammation, promote cell proliferation, alter host cell metabolism, and provide a microenvironment that facilitates cancer development.

Colorectal adenomas are considered precursor lesions of most cases of colorectal carcinoma. Advanced adenoma can be defined as a subset of adenoma in which the lesion size measures 10 mm or more and contains a substantially villous component or high-grade dysplasia. Only about 1-10% of people with adenomas develop colorectal carcinoma, while significantly more advanced adenoma patients eventually advance to colorectal carcinoma. For example, projections of 10 year cumulative risk for advanced adenoma progressing to colorectal cancer increase from 25.4% at age 55 years to 42.9% at age 80 years in women, and from 25.2% at age 55 years to 39.7% at age 80 years in men. Early detection and removal of advanced adenomas can dramatically decrease the incidence of colorectal carcinoma.

As with many other complex diseases, the susceptibility and progression of cancer are primarily influenced by gene-environment interactions. Tremendous progress has been made to explore the genetics and the molecular mechanisms that underlie carcinogenesis. The understanding of environmental factors that influence cancer susceptibility and progression, however, is still very limited. The microbiota is composed of bacteria, archaea, eukaryotes, and viruses that reside in different sites of the human body, including the gut and circulating blood. The microbiota is an example of an environmental factor that can influence carcinogenesis.

### I. Samples

In some embodiments, the present disclosure provides a system, method, or kit that includes or uses one or more biological samples. The one or more samples used herein may comprise any substance containing or presumed to contain nucleic acids. A sample can include a biological sample obtained from a subject. In some embodiments, a biological sample is a liquid sample. In some embodiments, a liquid sample is derived from whole blood, plasma, serum, ascites, cerebrospinal fluid, sweat, urine, tears, saliva, buccal sample, cavity rinse, or organ rinse. In some embodiments, a liquid sample is an essentially cell-free liquid sample or cell-free nucleic acid (cfNA), such as cell-free DNA (cfDNA). Non-limiting examples of cfNA can be found in fluids including, but not limited to plasma, serum, sweat, plasma, urine, sweat, tears, saliva, sputum, and cerebrospinal fluid. For example, a sample can be cfDNA.

In some embodiments, less than about 1 pg, less than about 5 pg, less than about 10 pg, less than about 20 pg, less than about 30 pg, less than about 40 pg, less than about 50 pg, less than about 100 pg, less than about 200 pg, less than about 500 pg, less than about 1 ng, less than about 5 ng, less than about 10 ng, less than about 20 ng, less than about 30 ng, less than about 40 ng, less than about 50 ng, less than about 100 ng, less than about 200 ng, less than about 500 ng, less than about 1 μg, less than about 5 μg, less than about 10 μg, less than about 20 μg, less than about 30 μg, less than about 40 μg, less than about 50 μg, less than about 100 μg, less than about 200 μg, less than about 500 μg, or less than about 1 mg of nucleic acids are obtained from the sample for analysis. In some cases, about 1 pg to about 5 pg, about 5 pg to about 10 pg, about 10 pg to about 100 pg, about 100 pg, to about 1 ng, about 1 ng to about 5 ng, about 5 ng to about 10 ng, about 10 ng to about 100 ng, about 100 ng to about 100 μg of nucleic acids are obtained from the sample for analysis.

In some embodiments, the methods described herein are used to detect and/or quantify nucleic acid sequences that correspond to a microbe of interest, or a microbiome of organisms. The methods described herein can analyze at least 1; at least 2; at least 3; at least 4; at least 5; at least 10; at least 20; at least 50; at least 100; at least 200; at least 500; at least 1,000; at least 2,000; at least 5,000; at least 10,000; at least 20,000; at least 50,000; at least 100,000; at least 200,000; at least 300,000; at least 400,000; at least 500,000; at least 600,000; at least 700,000; at least 800,000; at least 900,000; at least 10⁶; at least 5×10⁶; at least 10⁷; at least 5×10⁷; at least 10⁸; at least 5×10⁸; at least 10⁹; or more sequence reads.

In some embodiments, the methods described herein are used to detect and/or quantify gene expression, e.g., by determining the presence of mRNA from a microorganism in relation to DNA from that microorganism. In some embodiments, the methods described herein provide high discriminative and quantitative analysis of multiple genes. The methods described herein can discriminate and quantitate the expression of at least 1; at least 2; at least 3; at least 4; at least 5; at least 10; at least 20; at least 50; at least 100; at least 200; at least 500; at least 1,000; at least 2,000; at least 5,000; at least 10,000; at least 20,000; at least 50,000; at least 100,000; or more different target nucleic acids.

In one embodiment, a sample containing cell-free nucleic acids is obtained from a subject. Such subject can be a human, a domesticated animal, such as a cow, chicken, pig, horse, rabbit, dog, cat, goat, etc. In some embodiments, the cells used in methods of the present disclosure are taken from a patient. Samples include, for example, the acellular fraction of whole blood, sweat, tears, saliva, ear flow, sputum, lymph, bone marrow suspension, lymph, urine, saliva, semen, vaginal flow, cerebrospinal fluid, brain fluid, ascites, milk, secretions of the respiratory, intestinal or genitourinary tracts fluid, a lavage of a tissue or organ (e.g., lung) or tissue which has been removed from organs, such as breast, lung, intestine, skin, cervix, prostate, pancreas, heart, liver, and stomach. Such samples can be separated by centrifugation, elutriation, density gradient separation, apheresis, affinity selection, panning, FACS, centrifugation with Hypaque, etc. Once a sample is obtained, it can be used directly, frozen, or maintained in appropriate culture medium for short periods of time.

To obtain a blood sample, various techniques may be used, e.g., a syringe or other vacuum suction device. A blood sample can be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount can vary depending upon subject size and the condition being screened. In some embodiments, at least about 10 mL, at least about 5 mL., at least about 1 mL, at least about 0.5 mL, at least about 250 μL, at least about 200 μL, at least about 150 μL, at least about 100 μL, at least about 50 μL, at least about 40 μL, at least about 30 μL, at least about 20 μL, at least about 10 μL, at least about 9 μL, at least about 8 μL, at least about 7 μL, at least about 6 μL, at least about 5 μL, at least about 4 μL, at least about 3 μL, at least about 2 μL, or at least about 1 μL of a sample is obtained. In some embodiments, about 1 μL to about 50 μL, about 2 μL to about 40 μL, about 3 μL to about 30 μL, or about 4 μL to about 20 μL of sample is obtained. In some embodiments, more than about 5 μL, more than about 10 μL, more than about 15 μL, more than about 20 μL, more than about 25 μL, more than about 30 μL, more than about 35 μL, more than about 40 μL, more than about 45 μL, more than about 50 μL, more than about 55 μL, more than about 60 μL, more than about more than about 65 μL, more than about 70 μL, more than about 75 μL, more than about 80 μL, more than about 85 μL, more than about 90 μL, more than about 95 μL, or more than about 100 μL of a sample is obtained.

The method of the present disclosure may further comprise preparing a cell-free fraction from a biological sample. The cell-free fraction may be prepared using various techniques. For example, a cell-free fraction of a blood sample may be obtained by centrifuging the blood sample for about 3 min to about 30 min, preferably about 3 min to about 15 min, more preferably about 3 min to about 10 min, or more preferably about 3 min to about 5 min, at a low speed of about 200 g to about 20,000 g, preferably about 200 g to about 10,000 g, more preferably about 200 g to about 5,000 g, or more preferably about 350 g to about 4,500 g. The biological sample may be obtained by ultrafiltration in order to separate the cells and their fragments from a cell-free fraction comprising soluble DNA or RNA. Ultrafiltration may be carried out using a 0.22 μm membrane filter.

In some embodiments, a biological sample can include a solid biological sample. In some embodiments, a biological sample can be free of fecal matter. In some embodiments, a sample can include in vitro cell culture constituents. Cell culture constituents can include, for example, conditioned medium from cell growth in a cell culture medium, recombinant cells, and cell components. In some embodiments, a sample can include a single cell, a cancer cell, a circulating tumor cell, a cancer stem cell, white blood cells, red blood cells, lymphocytes, and the like. In some embodiments, a sample can include a plurality of cells. In some embodiments, a sample can contain about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or 100% tumor cells. In some embodiments, a subject can be suspected to harbor a solid tumor or known to harbor a solid tumor. In some embodiments, a subject can have previously harbored a solid tumor.

The sample may be taken before and/or after treatment of a subject with a disease or disorder. Samples may be obtained from a subject during a treatment or a treatment regime.

Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample can be taken at a first time point and sequenced, and then another sample can be taken at a subsequent time point and sequenced. Such methods can be used, for example, for longitudinal monitoring purposes to track the development or progression of a disease. In some embodiments, the progression of a disease can be tracked before treatment, after treatment, or during the course of treatment, to determine the treatment's effectiveness. For example, a method as described herein can be performed on a subject prior to, and after, treatment with a PD-1 immunotherapy to measure the disease's progression or regression in response to the immunotherapy.

After obtaining a sample from the subject, the sample may be processed to generate datasets indicative of a disease or disorder of the subject. For example, a presence, absence, or quantitative assessment of cell-free nucleic acid molecules of the sample at a panel of cancer-associated genomic loci or microbiome-associated loci may be indicative of a cancer of the subject. Processing the sample obtained from the subject may comprise (i) subjecting the sample to conditions that are sufficient to isolate, enrich, or extract a plurality of cell-free nucleic acid molecules, and (ii) assaying the plurality of cell-free nucleic acid molecules to generate the dataset (e.g., nucleic acid sequences).

In some embodiments, a plurality of cell-free nucleic acid molecules is extracted from the sample and subjected to sequencing to generate a plurality of sequencing reads. The cell-free nucleic acid molecules may comprise cell-free ribonucleic acid (cfRNA) or cell-free deoxyribonucleic acid (cfDNA). The cell-free nucleic acid molecules (e.g., cfRNA or cfDNA) may be extracted from the sample by a variety of methods, such as a FastDNA Kit protocol from MP Biomedicals, a QIAamp DNA cell-free biological mini kit from Qiagen, or a cell-free biological DNA isolation kit protocol from Norgen Biotek. The extraction method may extract all cfRNA or cfDNA molecules from a sample. Alternatively, the extraction method may selectively extract a portion of cfRNA or cfDNA molecules from a sample. Extracted cfRNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of cancer-associated genomic loci or microbiome-associated loci. The probes may be nucleic acid primers. The probes may have sequence complementarity with nucleic acid sequences from one or more of the panel of cancer-associated genomic loci or microbiome-associated features. The panel of cancer-associated genomic loci or microbiome-associated loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct cancer-associated genomic loci or microbiome-associated loci.

The probes may be nucleic acid molecules (e.g., RNA or DNA) having sequence complementarity with nucleic acid sequences (e.g., RNA or DNA) of the one or more genomic loci (e.g., cancer-associated genomic loci). These nucleic acid molecules may be primers or enrichment sequences. The assaying of the sample using probes that are selective for the one or more genomic loci (e.g., cancer-associated genomic loci or microbiome-associated loci) may comprise use of array hybridization, polymerase chain reaction (PCR), or nucleic acid sequencing (e.g., RNA sequencing or DNA sequencing).

The assay readouts may be quantified at one or more genomic loci (e.g., cancer-associated genomic loci) to generate the data indicative of the disease or disorder. For example, quantification of array hybridization or polymerase chain reaction (PCR) corresponding to a plurality of genomic loci (e.g., cancer-associated genomic loci or microbiome-associated loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

The intestinal microbiota of humans is dominated by species found within two bacterial phyla: members of the Bacteroides and Firmicutes make up >90% of the bacterial population. Actinobacteria (e.g., members of the Bifidobacterium genus) and Proteobacteria among several other phyla are less prominently represented to include >1000 prevalent bacterial species that confer a common core yet substantial inter-individual variability in the metagenome.

Common species of interest include prominent or less abundant members of this community, and may comprise, without limitation, Bacteroides thetaiotaomicron; Bacteroides caccae; Bacteroides fragilis; Bacteroides melaninogenicus; Bacteroides oralis; Bacteroides uniformis; Lactobacillus; Clostridium perfringens; Clostridium septicum; Clostridium tetani; Bifidobacterium bifidum; Staphylococcus aureus; Enterococcus faecalis; Escherichia coli; Salmonella enteritidis; Klebsiella sp.; Enterobacter sp.; Proteus mirabilis; Pseudomonas aeruginosa; Peptostreptococcus sp.; Peptococcus sp., Faecalibacterium sp; Roseburia sp.; Ruminococcus sp.; Dorea sp.; Alistipes sp.; etc.

In the skin microbiome, most bacteria fall into four different phyla: Actinobacteria, Firmicutes, Bacteroidetes, and Proteobacteria. Microorganisms that are generally regarded as skin colonizers include coryneforms of the phylum Actinobacteria (the genera Corynebacterium, Propionibacterium, such as Propionibacterium acnes; and Brevibacterium), the genus Micrococcus and Staphylococcus spp. The most commonly isolated fungal species are Malassezia spp., which are especially prevalent in sebaceous areas. The Demodex mites (such as Demodex folliculorum and Demodex brevis) may also be present. Other types of fungi that are thought to grow on the skin, include Debaryomyces and Cryptococcus spp. As non-commensals, burn wounds commonly become infected with S. pyogenes, Enterococcus spp., or Pseudomonas aeruginosa, and can also become infected with fungi and/or viruses. S. epidermidis is a very common skin commensal, but it is also the most frequent cause of hospital-acquired infection (HAI) on in-dwelling medical devices such as catheters or heart valves.

In various embodiments, the systems and methods disclosed herein comprise analyzing the taxonomic community composition of microbiota using sequencing results of cfDNA derived from the subjects. The taxonomic community can include one or more of the following microbes: Abiotrophia, Abiotrophia defectiva, Acidobacteria, Acidovorax, Acinetobacter, Acetanaerobacteria, Actinobacteria, Actinomycetes, Aeromonas, Agrobacterium, Akkermansia, Alistipes, Allobaculum, Aquabacterium, Azonexus, Bacillaceae_1, Bacteroides, Bacteroidetes, Bifidobacterium, Bifidobacterium bifidum, Bryantella, Catonella, Carnobacteriaceae_1, Chryseobacterium, Chryseomonas, Cloacibacterium, Clostridiales, Clostridium, Clostridium difficile, Clostridium tetani, Coriobacterineae, Corynebacteria, Comamonas, Cyanobacteria, Dechloromonas, Delftia, Enterobacter, Enterobacteriaceae, Enterococcus faecalis, Escherichia coli, Erwinia, Exiguobacterium, Firmicutes, Flavimonas, Fusobacteria, Gp1, Gp2, Haemophilus influenza, Helicobacter, Hoidemania, Klebsiella, Klebsiella bacterium, Lachnospiraceae incertae sedis, Lactobacillus, Lactococcus, Leuconostoc, Methylobacterium, Micrococcineae, Mycobacteria, Neisseria, Neisseria meningitides, Novosphingobium, Oligotropha, Pantoea, Paiudibacter, Proteobacteria, Proteus, Pseudomonas, Pseudomonas aeruginosa, Pseudoxanthomonas, Raistonia, Rikeneia, Roseburia, Rubrobacterineae, Serratia, Shinella, Sphingobium, Spirochetes, Sporobacter, Staphylococcus, Staphylococcus aureus, Staphylococcus epidermidis, Staphylococcus mitis, Stenotrophomonas, Streptococcus mutans, Streptococcus pneumoniae, Streptococcus pyogenes, Streptococcus salivarius, Stenotrophomonas, Succinivibrio, Sutterella, Syntrophococcus, Turicibacter, Variovorax, Verrucomicrobia, and Weissella.

In one embodiment, the sequences are mapped to species of microbiota selected from Propionibacterium spp., Candidatus Zinderia spp., Dasheen mosaic virus, Vicia cryptic virus, Comamonas spp., Caulobacter spp., Acinetobacter spp., Burkholdreia spp., Micrococcus spp., Candidatus Sulcia spp., Torque teno virus, Polaromonas spp., Pseudomonas spp., Acinetobacter spp., Cupriavidus spp., Dietzia spp., Neisseria spp., Propionibacterium spp., Stenotrophomonas spp., and combinations thereof.

In one embodiment, the sequences are mapped to species of microbiota selected from Propionibacterium acnes, Candidatus Zinderia insecticola, Dasheen mosaic virus, Vicia cryptic virus, Comamonas spp., Caulobacter spp., Acinetobacter spp., Burkholdreia spp., Micrococcus luteus, Candidatus Sulcia muelleri, Torque teno virus, Polaromonas spp., Pseudomonas spp., Acinetobacter johnsonii, Cupriavidus spp., Dietzia spp., Neisseria spp., Propionibacterium granulosum, Stenotrophomonas maltophilia, and combinations thereof.

In one embodiment, the sequences are mapped to species of microbiota selected from Propionibacterium acnes, Candidatus Zinderia insecticola, Dasheen mosaic virus, Vicia cryptic virus, and combinations thereof.

In one embodiment, the sequences are mapped to species of microbiota selected from Propionibacterium acnes, Candidatus Zinderia insecticola, Dasheen mosaic virus, Vicia cryptic virus, Comamonas spp., Caulobacter spp., Acinetobacter spp., and combinations thereof.

In one embodiment, the sequences are mapped to species of microbiota selected from Propionibacterium acnes, Candidatus Zinderia insecticola, Dasheen mosaic virus, Vicia cryptic virus, Comamonas spp., Caulobacter spp., Acinetobacter spp., Burkholdreia spp., Micrococcus luteus, Candidatus Sulcia muelleri, Torque teno virus, and combinations thereof.

In various embodiments, cfDNA sequences derived from microbiome species Fusobacterium nucleatum, Bacteroides clarus, Roseburia intestinalis, Clostridium hathewayi, and/or one undefined species (m7) are significantly different in CRC patients in comparison to healthy controls as previously shown in duplex-qPCR assays. In various embodiments, the presence of cfDNA derived from these species and increased relative abundance of cfDNA sequences derived from these species contribute to the stratification of subjects with CRC in the methods described herein.

### II. Nucleic Acids

In some embodiments, the present disclosure provides a system, method, or kit that includes or uses nucleic acids. In some embodiments, nucleic acids containing germline sequences can be extracted from a biological sample from a subject. In some embodiments, the biological sample is a solid tissue. The biological sample can be tissue, such as normal or healthy tissue from the subject. The biological sample can be a liquid sample, including, for example, blood, buffy coat from blood (which can include lymphocytes), saliva, or plasma.

In some embodiments, nucleic acids that contain somatic variants can be extracted from a biological sample of a subject. In some embodiments, a biological sample can include a solid tissue, a primary tumor, a metastasis tumor, a polyp, or an adenoma. In some embodiments, a biological sample can include a liquid sample, urine, saliva, cerebrospinal fluid, plasma, or serum. In some embodiments, the liquid is a cell-free liquid. In some embodiments, cells from a liquid sample can be enriched or isolated. In some embodiments, the sample can include cell-free nucleic acid, e.g., DNA or RNA. In some embodiments, nucleic acids described herein can include RNA, DNA, genomic DNA, single-stranded DNA (ssDNA), double-stranded DNA (dsDNA), mitochondrial DNA, viral DNA, synthetic DNA, or cDNA reverse transcribed from RNA. In various embodiments, the nucleic acid can be single stranded or double stranded. In one embodiment, the nucleic acid is ssDNA to increase the number of cfNA microbiome sequence reads.

In some embodiments, the terms “polynucleotides”, “nucleic acid”, and “oligonucleotides” can be used interchangeably. These terms can refer to a polymeric form of nucleotides of any length, either deoxyribonucleotides or ribonucleotides, or analogs thereof. In some embodiments, polynucleotides have any three-dimensional structure. In some embodiments, polynucleotides can perform any function, known or unknown. Non-limiting examples of polynucleotides include coding regions of a gene or gene fragment, non-coding regions of a gene or gene fragment, loci (locus) defined from linkage analysis, exons, introns, messenger RNA (mRNA), transfer RNA, ribosomal RNA, ribozymes, complementary DNA (cDNA), recombinant polynucleotides, branched polynucleotides, plasmids, vectors, isolated DNA of any sequence, isolated RNA of any sequence, nucleic acid probes, and primers. In some embodiments, RNA can be reverse transcribed to generate cDNA. In some embodiments, a polynucleotide can include modified nucleotides, such as methylated nucleotides and nucleotide analogs. If present, modifications to the nucleotide structure can be imparted before or after assembly of the polymer. In some embodiments, a sequence of nucleotides can be interrupted by non-nucleotide components. In some embodiments, a polynucleotide is further modified after polymerization, such as by conjugation with a labeling component.

### III. Nucleic Acid Sequencing

Sequencing reads can be obtained from various sources including, for example, whole genome sequencing, whole exome sequencing, targeted sequencing, next-generation sequencing, pyrosequencing, sequencing-by-synthesis, ion semiconductor sequencing, tag-based next generation sequencing, semiconductor sequencing, single-molecule sequencing, nanopore sequencing, sequencing-by-ligation, sequencing-by-hybridization, Digital Gene Expression (DGE), massively parallel sequencing, Clonal Single Molecule Array (Solexa/Illumina), sequencing using PacBio, Sequencing by Oligonucleotide Ligation and Detection (SOLiD). In some embodiments, a sample comprising cfDNA is free of fecal matter.

In various embodiments, the sequencing reads are obtained via a next-generation sequencing method or a next-next-generation sequencing method.

In various embodiments, the sequencing reads are obtained via at least one system selected from the group consisting of Hiseq 2000, SOLID, 454, and True Single Molecule Sequencing.

In some embodiments, sequencing comprises modification of a nucleic acid molecule or fragment thereof, for example, by ligating a barcode, a unique molecular identifier (UMI), or anothertag to the nucleic acid molecule or fragment thereof. Ligating a barcode, UMI, or tag to one end of a nucleic acid molecule or fragment thereof may facilitate analysis of the nucleic acid molecule or fragment thereof following sequencing. In some embodiments, a barcode is a unique barcode (e.g., a UMI). In some embodiments, a barcode is non-unique, and barcode sequences can be used in connection with endogenous sequence information such as the start and stop sequences of a target nucleic acid (e.g., the target nucleic acid is flanked by the barcode and the barcode sequences, in connection with the sequences at the beginning and end of the target nucleic acid, creates a uniquely tagged molecule).

A barcode, UMI, or tag can be a known sequence used to associate a polynucleotide or fragment thereof with an input or target nucleic acid molecule or fragment thereof. A barcode, UMI, or tag may comprise natural nucleotides or non-natural (e.g., modified) nucleotides (e.g., as described herein). A barcode sequence can be contained within an adapter sequence such that the barcode sequence can be contained within a sequencing read. A barcode sequence may comprise at least 4, 5, 6, 7, 8, 9, 10, 11, 12, 13, 14, 15, 16, or more nucleotides in length. In some cases, a barcode sequence can be of sufficient length and can be sufficiently different from another barcode sequence to allow the identification of a sample based on a barcode sequence with which it is associated. A barcode sequence, or a combination of barcode sequences, can be used to tag and subsequently identify an “original” nucleic acid molecule or fragment thereof (e.g., a nucleic acid molecule or fragment thereof present in a sample from a subject). In some cases, a barcode sequence, or a combination of barcode sequences, is used in conjunction with endogenous sequence information to identify an original nucleic acid molecule or fragment thereof. For example, a barcode sequence, or a combination of barcode sequences, can be used with endogenous sequences adjacent to a barcode, UMI, or tag (e.g., the beginning and end of the endogenous sequences) and/or with the length of the endogenous sequence.

Processing a nucleic acid molecule or fragment thereof may comprise performing nucleic acid amplification. For example, any type of nucleic acid amplification reaction can be used to amplify a target nucleic acid molecule or fragment thereof and generate an amplified product. Non-limiting examples of nucleic acid amplification methods include reverse transcription, primer extension, polymerase chain reaction (PCR), ligase chain reaction, asymmetric amplification, rolling circle amplification, and multiple displacement amplification (MDA). Examples of PCR include, but are not limited to, quantitative PCR, real-time PCR, digital PCR, emulsion PCR, hot start PCR, multiplex PCR, asymmetric PCR, nested PCR, and assembly PCR. Nucleic acid amplification may involve one or more reagents such as one or more primers, probes, polymerases, buffers, enzymes, and deoxyribonucleotides. Nucleic acid amplification can be isothermal or may comprise thermal cycling. Thermal cycling may comprise two or more discrete temperature steps. A temperature step can be associated with a particular process such as initialization, denaturation, annealing, and extension. A single thermal cycle may comprise denaturation, annealing, and extension. Multiple thermal cycles can be performed to amplify a nucleic acid molecule or fragment thereof to a detectable level.

In various embodiments, a quantitative polymerase chain reaction (qPCR) assay allows the detection of both internal control and target in the same reaction for each sample, saving both reagents and samples, and producing more reliable data.

In one embodiment, target marker abundance is calculated relative to total bacterial nucleic acid content by the ΔCp method. In particular, DNA template concentration may be limited (<10 ng/μL) to avoid inhibitory effects caused by fecal DNA and may have a minimum quantity (>0.1 ng/μL) to avoid false-negative assessments of the targets using our duplex qPCR assays. A good correlation may be achieved in the quantification of bacterial candidates by metagenomics approach and qPCR assays. Therefore, the duplex-qPCR assays are reliable, convenient, and of excellent clinical application value in the quantitative detection of target bacteria.

The present disclosure provides methods comprising high-throughput sequencing of a cell-free nucleic acid sample from a subject, followed by bioinformatics analysis to determine the presence and prevalence of microbial sequences, which sequences may be from indigenous organisms, e.g., the normal microbiome of gut, skin, etc., or may be non-indigenous, e.g., opportunistic, pathogenic, etc. infections. Analysis may be performed for the complete microbiome, or for components thereof, for example the virome, bacterial microbiome, fungal microbiome, protozoan microbiome, etc. Examples of nucleic acids include, but are not limited to double-stranded DNA, single-stranded DNA, single-stranded DNA hairpins, DNA RNA hybrids, RNA (e.g., mRNA or miRNA), and RNA hairpins. In some embodiments, the nucleic acid is DNA. In some embodiments, the nucleic acid is RNA. For instance, cell-free RNA and DNA are present in human plasma.

Genotyping microbiome nucleic acids, and/or detection, identification, and/or quantitation of the microbiome-specific nucleic acids generally include an initial step of amplification of the sample, although there may be instances where sufficient cell-free nucleic acids are available and can be directly sequenced. When the nucleic acid is RNA, the amplification step may be preceded by a reverse transcriptase reaction to convert the RNA into DNA. Preferably, the amplification is unbiased, that is the primers for amplification are universal primers, or adaptors are ligated to the nucleic acids being analyzed, and amplification primers are specific for the adaptors. Examples of PCR techniques include, but are not limited to, hot start PCR, nested PCR, in situ polonony PCR, in situ rolling circle amplification (RCA), bridge PCR, picotiter PCR, and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR), and nucleic acid based sequence amplification (NABSA). Other amplification methods that may be used to amplify specific polymorphic loci include those described in, for example, U.S. Pat. Nos. 5,242,794; 5,494,810; 4,988,617; and 6,582,938, each of which is hereby incorporated in its entirety.

Following amplification, the amplified nucleic acid may be sequenced. Sequencing can be accomplished using high-throughput systems, some of which allow detection of a sequenced nucleotide immediately after or upon its incorporation into a growing strand, e.g., detection of sequence in real time or substantially real time. In some cases, high-throughput sequencing generates at least 1,000, at least 5,000, at least 10,000, at least 20,000, at least 30,000, at least 40,000, at least 50,000, at least 100,000, or at least 500,000 sequence reads per hour; with each read being at least 50, at least 60, at least 70, at least 80, at least 90, at least 100, at least 120, or at least 150 bases per read. Sequencing can be performed using nucleic acids described herein such as genomic DNA, cDNA derived from RNA transcripts, or RNA as a template.

In some embodiments, high-throughput sequencing involves the use of technology available by Helicos Biosciences Corporation (Cambridge, Mass.) such as the Single Molecule Sequencing by Synthesis (SMSS) method. SMSS is unique because it allows for sequencing an entire genome with no pre amplification step needed. Thus, distortion and nonlinearity in the measurement of nucleic acids are reduced. SMSS is described, for example, in US Pat. Publication Nos. 20060024711; 20060024678; 20060012793; 20060012784; and 20050100932, each of which is hereby incorporated in its entirety.

In some embodiments, high-throughput sequencing involves the use of technology available by 454 Lifesciences, Inc. (Branford, Conn.) such as the Pico Titer Plate device, which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described, for example, in US Pat. Publication Nos. 20020012930; 20030058629; 20030100102; 20030148344; 20040248161; 20050079510, 20050124022; and 20060078909; each of which is hereby incorporated in its entirety.

In some embodiments, high-throughput sequencing is performed using Clonal Single Molecule Array (Solexa, Inc.) or sequencing-by-synthesis (SBS) utilizing reversible terminator chemistry. These technologies are described, for example, in U.S. Pat. Nos. 6,969,488; 6,897,023; 6,833,246; 6,787,308; and US Pat. Publication Nos. 20040106130; 20030064398; and 20030022207; each of which is hereby incorporated in its entirety.

In some embodiments, high-throughput sequencing of RNA or DNA can take place using AnyDot.chips (Genovoxx, Germany), which allows for the monitoring of biological processes, e.g., miRNA expression or allele variability (SNP detection). In particular, the AnyDot.chips allow for 10×-50× enhancement of nucleotide fluorescence signal detection. AnyDot.chips and methods for using them are described in part in International Pat. Publication Nos. WO 02088382, WO 03020968, WO 03031947, WO 2005044836, PCTEP 05105657, PCMEP 05105655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301; each of which is hereby incorporated in its entirety.

Other high-throughput sequencing systems include those disclosed in US Pat. Publication Nos. 20030044781 and 2006/0078937; each of which is hereby incorporated in its entirety. Overall such systems may involve sequencing a target nucleic acid molecule having a plurality of bases by the temporal addition of bases via a polymerization reaction that is measured on a molecule of nucleic acid, e.g., the activity of a nucleic acid polymerizing enzyme on the template nucleic acid molecule to be sequenced is followed in real time. Sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. A plurality of labeled types of nucleotide analogs are provided proximate to the active site, with each distinguishable type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended, and the sequence of the target nucleic acid is determined.

In some embodiments, shotgun sequencing is performed. In shotgun sequencing, DNA is broken up randomly into numerous small segments, which are sequenced using the chain termination method to obtain reads. Multiple overlapping reads for the target DNA are obtained by performing several rounds of this fragmentation and sequencing. Computer programs then use the overlapping ends of different reads to assemble them into a continuous sequence.

### IV. Analysis and Machine Learning Methods

The taxonomic community composition of microbiota in cfDNA can be identified by applying sequence alignment methods to map unmapped reads from whole genome sequencing of a human reference genome on the taxonomic-specific genetic markers summarized from the NIH Human Microbiome Project. The taxonomic community composition of microbiota can be determined by estimating the normalized number of reads that map to the whole taxonomic-specific genetic markers. Non-limiting examples of sequence alignment methods include Metagenomic Phylogenetic Analysis (for example, MetaPhlAn2), BLAT, Burrows-Wheeler Aligner (BWA), Bowtie, Bowtie2, Bfast, BioScope, CLC bio, Cloudburst, Eland/Eland2, GenomeMapper, GnuMap, Karma, MAQ, MOM, Mosaik, MrFAST/MrsFAST, NovoAlign, PASS, PerM, RazerS, RMAP, SSAHA2, Segemehl, SeqMap, SHRiMP, Slider/SliderIl, Srprism, Stampy, vmatch, ZOOM, and the SOAP/SOAP2 alignment tool.

### A. Sample Features

In various embodiments, to find correlations between microbiota composition and diseases or conditions, feature matrices are generated to compare and distinguish samples obtained from subjects with known conditions (positive samples) from samples obtained from healthy subjects, or subjects who do not have any of the known indications (negative or control samples).

As used herein, as it relates to machine learning and pattern recognition, the term “feature” refers to an individual measurable property or characteristic of a phenomenon being observed, or a subset thereof. Features may be numeric, but may also include structural features such as strings and graphs, such as those used in syntactic pattern recognition. For example, features may include characters or strings of characters representing one or more contiguous nucleotides of a polynucleotide. The concept of “feature” is related to that of explanatory variable used in statistical techniques such as linear regression.

In one embodiment, the features are inputted into a feature matrix for machine learning analysis.

For a plurality of assays, the system identifies feature sets to input to a machine learning model. The system performs an assay on each molecule class and forms a feature vector from the measured values. The system inputs the feature vector into the machine learning model and obtains an output classification, prediction, or likelihood of whether the biological sample has a specified property.

In one embodiment, the machine learning model produces a classifier capable of distinguishing between two groups or classes of individuals or features in a population of individuals or features of the population. In one embodiment, the classifier is a trained machine learning classifier.

In one embodiment, the informative loci or features of biomarkers in a cancer tissue are assayed to form a profile. Receiver operating characteristic (ROC) curves may be useful for plotting the performance of a particular feature (e.g., any of the biomarkers described herein and/or any item of additional biomedical information) in distinguishing between two populations (e.g., individuals responding and not responding to a therapeutic agent). Typically, the feature data across the entire population (e.g., the cases and controls) are sorted in ascending order based on the value or importance of individual features.

In some embodiments, the condition (e.g., disease or disorder) is a cancer (e.g., advanced adenoma (AA), colorectal carcinoma), or inflammatory bowel disease. In one embodiment, the feature matrix normalizes the number of reads from each taxonomic level and estimates the relative abundance of taxonomic community composition of the microbiota.

In various embodiments, the taxonomic community is a kingdom, a phylum, a class, an order, a family, a genus, or a species of the microbiota.

In various embodiments, the feature is the relative abundance of sequences in one or more of the communities.

As used herein, the term “relative abundance” refers to the abundance of a target nucleic acid or nucleic acids compared to a reference population, such as the total non-matched non-human nucleic acid population. For example, the relative abundance of a microbiota species may be estimated by summing the abundance of the genomes belonging to the non-human nucleic acid complement in cfDNA.

B. Data analysis

In some embodiments, the present disclosure provides a system, method, or kit having data analysis realized in software application, computing hardware, or both. In various embodiments, the analysis application or system includes at least a data receiving module, a data pre-processing module, a data analysis module (which can operate on one or more types of genomic data), a data interpretation module, or a data visualization module. In one embodiment, the data receiving module can comprise computer systems that connect laboratory hardware or instrumentation with computer systems that process laboratory data. In one embodiment, the data pre-processing module can comprise hardware systems or computer software that performs operations on the data in preparation for analysis. Examples of operations that can be applied to the data in the pre-processing module include affine transformations, denoising operations, data cleaning, reformatting, or subsampling. A data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

After generating the feature sets from datasets obtained using one or more assays, a trained algorithm may be used to process one or more of the feature sets to identify or assess the condition (e.g., diseases or disorder, such as CRC or AA). For example, the trained algorithm may be used to apply a machine learning classifier to a plurality of microbiome-associated features (e.g., microbiome species and abundance of microbiome elements) that are associated with two or more classes of individuals inputted into a machine learning model, in order to classify a subject into one of the two or more classes of subjects. For example, the trained algorithm may be used to apply a machine learning classifier to a plurality of microbiome-associated features (e.g., microbiome species and abundance of microbiome elements) that are associated with subjects with known conditions (e.g., a disease or disorder, such as CRC or AA) and subjects not having the condition (e.g., healthy subjects, or subjects who do not have any of the known indications), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as CRC or AA) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., microbiome-associated features, such as microbiome species and abundance of microbiome elements) and to produce or output one or more output values based on the plurality of input variables or features (e.g., microbiome-associated features, such as microbiome species and abundance of microbiome elements). The plurality of input variables or features may comprise one or more datasets indicative of the presence (e.g., positive test result) or absence (e.g., negative test result) of one or more conditions (e.g., a disease or disorder, such as CRC or AA). For example, an input variable or feature may comprise a number of sequences corresponding to or aligning to each of the plurality of cancer-associated genomic loci or microbiome-associated features.

The plurality of input variables or features may also include clinical information of a subject, such as health data. For example, the health data of a subject may comprise one or more of: a diagnosis of one or more conditions (e.g., a disease or disorder, such as CRC or AA), a prognosis of one or more conditions (e.g., a disease or disorder, such as CRC or AA), a risk of having one or more conditions (e.g., a disease or disorder, such as CRC or AA), a treatment history of one or more conditions (e.g., a disease or disorder, such as CRC or AA), a history of previous treatment for one or more conditions (e.g., a disease or disorder, such as CRC or AA), a history of prescribed medications, a history of prescribed medical devices, age, height, weight, sex, smoking status, and one or more symptoms of the subject. For example, the disease or disorder may comprise one or more of: CRC, AA, and IBD. In some embodiments, the one or more symptoms comprise chronic fatigue, weight loss, nausea, and insomnia.

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as CRC or AA) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MM) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject (e.g., an estimated expected or average progression-free survival (PFS) or overall survival (OS) of the subject in number of days, weeks, or months). Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1},{positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as CRC or AA) of the subject and may comprise, for example, an indication of an estimated expected or average progression-free survival (PFS) or overall survival (OS) of the subject in number of days, weeks, or months. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as CRC or AA), thereby assigning the subject to a class of subjects receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of subjects receiving a negative test result. In this case, a single cutoff value of 50% is used to classify samples into one of the two possible binary output values or classes of subjects (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as CRC or AA) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more conditions (e.g., a disease or disorder, such as CRC or AA) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as CRC or AA) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as CRC or AA) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of subjects (e.g., corresponding to outcome groups of subjects having “low risk,” “intermediate risk,” and “high risk” of having one or more conditions, such as a disease or disorder). Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of subjects, where n is any positive integer.

The trained algorithm may be trained with a plurality of independent training samples. Each of the independent training samples may comprise a sample containing cell-free nucleic acids from a subject, associated datasets obtained by assaying the cell-free nucleic acids of the sample (as described elsewhere herein), and one or more known output values or classes of subjects corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment (e.g., a surgery, a chemotherapy, a radiotherapy, or an immunotherapy) for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder). In some embodiments, the sample is independent of samples used to train the trained algorithm.

The trained algorithm may be trained with a first number of independent training samples associated with a presence of the condition (e.g., a disease or disorder) and a second number of independent training samples associated with an absence of the condition (e.g., a disease or disorder). The first number of independent training samples associated with presence of the condition (e.g., a disease or disorder) may be no more than the second number of independent training samples associated with absence of the condition (e.g., a disease or disorder). The first number of independent training samples associated with a presence of the condition (e.g., a disease or disorder) may be equal to the second number of independent training samples associated with an absence of the condition (e.g., a disease or disorder). The first number of independent training samples associated with a presence of the condition (e.g., a disease or disorder) may be greater than the second number of independent training samples associated with an absence of the condition (e.g., a disease or disorder).

The trained algorithm may comprise a classifier configured to identify the presence (e.g., positive test result) or absence (e.g., negative test result) of one or more conditions (e.g., a disease or disorder, such as CRC or AA) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as CRC or AA) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as CRC or AA) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as CRC or AA) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as CRC or AA) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as CRC or AA) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), or identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the subject classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of cancer-associated genomic loci or microbiome-associated features may be identified as most influential or most important to be included for making high-quality classifications or identifications of conditions (or sub-types of conditions). The panel of cancer-associated genomic loci or microbiome-associated features, or a subset thereof, may be ranked based on classification metrics indicative of each influence or importance of each subject cancer-associated genomic locus or microbiome-associated feature toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of cancer-associated genomic loci or microbiome-associated features) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as CRC or AA), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, or any combination thereof.

The feature sets (e.g., comprising quantitative measures of a panel of cancer-associated genomic loci or microbiome-associated features) may be analyzed and assessed (e.g., using a trained algorithm comprising one or more classifiers) over a duration of time to monitor a patient (e.g., a subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of cancer-associated genomic loci or microbiome-associated features) determined at each of the two or more time points.

In some embodiments, a difference in the feature sets (e.g., quantitative measures of a panel of cancer-associated genomic loci or microbiome-associated features) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of cancer-associated genomic loci or microbiome-associated features) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, or any combination thereof.

In some embodiments, a difference in the feature sets (e.g., quantitative measures of a panel of cancer-associated genomic loci or microbiome-associated features) determined between the two or more time points may be indicative of a prognosis of the condition of the subject.

In some embodiments, a difference in the feature sets (e.g., quantitative measures of a panel of cancer-associated genomic loci or microbiome-associated features) determined between the two or more time points may be indicative of the subject having an increased risk of the condition. For example, if the condition was detected in the subject both at an earlier time point and at a later time point, and if the difference is a negative difference (e.g., the quantitative measures of a panel of cancer-associated genomic loci or microbiome-associated features increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, or any combination thereof.

In some embodiments, a difference in the feature sets (e.g., quantitative measures of a panel of cancer-associated genomic loci or microbiome-associated features) determined between the two or more time points may be indicative of the subject having a decreased risk of the condition. For example, if the condition was detected in the subject both at an earlier time point and at a later time point, and if the difference is a positive difference (e.g., the quantitative measures of a panel of cancer-associated genomic loci or microbiome-associated features decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, or any combination thereof.

In some embodiments, a difference in the feature sets (e.g., quantitative measures of a panel of cancer-associated genomic loci or microbiome-associated features) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, or any combination thereof.

In some embodiments, a difference in the feature sets (e.g., quantitative measures of a panel of cancer-associated genomic loci or microbiome-associated features) determined between the two or more time points may be indicative of a non-efficacy of the course of treatment for treating the condition of the subject. For example, if the condition was detected in the subject both at an earlier time point and at a later time point, and if the difference is a negative or zero difference (e.g., the quantitative measures of a panel of cancer-associated genomic loci or microbiome-associated features increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, or any combination thereof.

In various embodiments, machine learning methods are applied to distinguish samples in a population of samples. In one embodiment, machine learning methods are applied to distinguish samples between healthy and advanced adenoma samples.

In one embodiment, the one or more machine learning operations used to train the microbiota prediction engine include one or more of: a generalized linear model, a generalized additive model, a non-parametric regression operation, a random forest (RF) classifier, a spatial regression operation, a Bayesian regression model, a time series analysis, a Bayesian network, a Gaussian network, a decision tree learning operation, an artificial neural network (e.g., a convolutional neural network (CNN), a deep neural network (DNN), or a deep convolutional neural network (DCNN)), a recurrent neural network (RNN), a reinforcement learning operation, linear or non-linear regression operations, a support vector machine (SVM), a clustering operation, and a genetic algorithm operation.

In various embodiments, computer processing methods are selected from logistic regression, linear regression, multiple linear regression (MLR), dimension reduction, partial least squares (PLS) regression, principal component regression, autoencoders, variational autoencoders, singular value decomposition, Fourier bases, wavelets, discriminant analysis, support vector machine, decision tree, classification and regression trees (CART), tree-based methods, random forest, gradient boost tree, logistic regression, matrix factorization, multidimensional scaling (MDS), dimensionality reduction methods, t-distributed stochastic neighbor embedding (t-SNE), multilayer perceptron (MLP), network clustering, neuro-fuzzy, and artificial neural networks (e.g., a convolutional neural network (CNN), a deep neural network (DNN), or a deep convolutional neural network (DCNN)).

In some embodiments, the methods disclosed herein can include computational analysis on nucleic acid sequencing data of samples from a subject or from a plurality of subjects. An analysis can identify a variant inferred from sequence data to identify sequence variants based on probabilistic modeling, statistical modeling, mechanistic modeling, network modeling, or statistical inferences. Non-limiting examples of analysis methods include principal component analysis (PCA), autoencoders, singular value decomposition (SVD), Fourier bases, wavelets, discriminant analysis, regression, support vector machines (SVM), tree-based methods, networks, matrix factorization, and clustering. Non-limiting examples of variants include a germline variation or a somatic mutation. In some embodiments, a variant can refer to an already-known variant. The already-known variant can be scientifically confirmed or reported in literature. In some embodiments, a variant can refer to a putative variant associated with a biological change. A biological change can be known or unknown. In some embodiments, a putative variant can be reported in literature, but not yet biologically confirmed.

Alternatively, a putative variant may not be reported in literature, but can be inferred based on a computational analysis disclosed herein. In some embodiments, germline variants can refer to nucleic acids that induce natural or normal variations.

Natural or normal variations can include, for example, skin color, hair color, and normal weight. In some embodiments, somatic mutations can refer to nucleic acids that induce acquired or abnormal variations. Acquired or abnormal variations can include, for example, cancer, obesity, conditions, symptoms, diseases, and disorders. In some embodiments, the analysis can include distinguishing between germline variants. Germline variants can include, for example, private variants and somatic mutations. In some embodiments, the identified variants can be used by clinicians or other health professionals to improve health care methodologies, accuracy of diagnoses, and cost reduction.

Also provided herein are improved methods and computing systems or software media that can distinguish among sequence errors in nucleic acid introduced through amplification and/or sequencing techniques, somatic mutations, and germline variants. Methods provided can include simultaneously calling and scoring variants from aligned sequencing data of all samples obtained from a patient.

Samples obtained from subjects other than the patient can also be used. Other samples can also be collected from subjects previously analyzed by a sequencing assay or a targeted sequencing assay (e.g., a targeted resequencing assay). Methods, computing systems, or software media disclosed herein can improve identification and accuracy of variations or mutations (e.g., germline or somatic, including copy number variations, single nucleotide variations, indels, a gene fusions), and lower limits of detection by reducing the number of false positive and false negative identifications.

In various embodiments, the features are ranked according to the importance in terms of prediction or classification. In various embodiments, Permutation Feature Importance (PFI) analysis is applied to identify the best performing models on each dataset. PFI is a method which assigns relative importance to input features based on the level of prediction after each feature random reshuffling. The larger the decrease in accuracy of prediction, the more important the input feature is.

In various embodiments, PFI identifies microbial elements in the sample that have increased importance to the predictive value of the classifier.

In various embodiments, the methods include a calibrating step including the steps of: obtaining data and calibrating preprocessed detected values by means of training a linear discriminant analysis classifier with known relative abundance of microbiota in a human subject and applying the trained classifier to the preprocessed detected value data set of a subject suspected of having CRC or AA and using the trained classifier to determine the presence of CRC or AA in a human subject.

In various embodiments, the calibrating comprises: a) mathematically preprocessing the at least one measured value in order to reduce technical errors in the measuring; b) selecting at least one suitable classifying algorithm from the group consisting of logistic regression, linear or quadratic discriminant analysis, perceptron, shrunken centroids regularized discriminant analysis, random forests, neural networks, Bayesian networks, hidden Markov models, support vector machines, generalized partial least squares, partitioning around medoids, inductive logic programming, generalized additive models, Gaussian processes, regularized least square regression, self-organizing maps, recursive partitioning and regression trees, k-nearest neighbor classifiers, fuzzy classifiers, bagging, boosting, and naive Bayes; and applying the selected classifier algorithm to preprocessed data of a); c) training the at least one suitable classifying algorithm of b) on at least one training data set containing preprocessed data from subjects divided into classes according to their asphyxia-related pathophysiological, physiological, prognostic, or responder conditions, in order to select a classifier function to map the preprocessed data to the conditions; and d) applying the trained at least one suitable classifying algorithm of c) to a preprocessed data set of a subject with unknown CRC pathophysiological, physiological, prognostic, or responder condition, and using the trained at least one suitable classifying algorithm to assign the subject into either CRC or AA groups in order to diagnose an asphyxia status of the subject.

### C. Classifier Generation

In one aspect, the present systems and methods provide a model or classifier generated based on feature information derived from microbiome sequence analysis from biological samples of cfDNA. The classifier forms part of a predictive engine for distinguishing groups in a population based on microbiome sequence features identified in biological samples such as cfDNA.

In one embodiment, a classifier is created by normalizing the microbiota information by formatting similar portions of the microbiota information into a unified format and a unified scale; storing the normalized microbiota information in a columnar database; training a microbiota prediction engine by applying one or more one machine learning operations to the stored normalized microbiota information, the microbiota prediction engine mapping, for a particular microbiota population, a combination of one or more features; applying the microbiota prediction engine to the accessed field information to identify a microbiome associated with a group; and classifying the subject into a group.

Specificity may refer to “the probability of a negative test among those who are free from the disease”. It equals a number of disease-free persons who tested negative divided by the total number of disease-free subjects.

In various embodiments, the model, classifier, or predictive test has a specificity of at least 40%, at least 45%, at least 50%, at least 55%, at least 60%, at least 65%, at least 70%, at least 75%, at least 80%, at least 85%, at least 90%, at least 95%, or at least 99%.

Sensitivity may refer to “the probability of a positive test among those who have the disease”. It equals a number of diseased subjects who tested positive divided by the total number of diseased subjects.

In various embodiments, the model, classifier, or predictive test has a sensitivity of at least 40%, at least 45%, at least 50%, at least 55%, at least 60%, at least 65%, at least 70%, at least 75%, at least 80%, at least 85%, at least 90%, at least 95%, or at least 99%.

In one embodiment, the group is selected from healthy (asymptomatic), IBD, AA, or CRC.

### D. Digital Processing Device

In some embodiments, the subject matter described herein can include a digital processing device or use of the same. In some embodiments, the digital processing device can include one or more hardware central processing units (CPU), graphics processing units (GPU), or tensor processing units (TPU) that carry out the device's functions. In some embodiments, the digital processing device can include an operating system configured to perform executable instructions. In some embodiments, the digital processing device can optionally be connected a computer network. In some embodiments, the digital processing device can be optionally connected to the Internet such that it accesses the World Wide Web. In some embodiments, the digital processing device can be optionally connected to a cloud computing infrastructure. In some embodiments, the digital processing device can be optionally connected to an intranet. In some embodiments, the digital processing device can be optionally connected to a data storage device.

Non-limiting examples of suitable digital processing devices include server computers, desktop computers, laptop computers, notebook computers, sub-notebook computers, netbook computers, netpad computers, set-top computers, handheld computers, Internet appliances, mobile smartphones, and tablet computers. Suitable tablet computers can include, for example, those with booklet, slate, and convertible configurations.

In some embodiments, the digital processing device can include an operating system configured to perform executable instructions. For example, the operating system can include software, including programs and data, which manages the device's hardware and provides services for execution of applications. Non-limiting examples of operating systems include Ubuntu, FreeBSD, OpenBSD, NetBSD, Linux, Apple Mac OS X Server®, Oracle® Solaris®, Windows Server®, and Novell® NetWare®. Non-limiting examples of suitable personal computer operating systems include Microsoft® Windows®, Apple® Mac OS X®, UNIX®, and UNIX-like operating systems such as GNU/Linux®. In some embodiments, the operating system can be provided by cloud computing, and cloud computing resources can be provided by one or more service providers.

In some embodiments, the device can include a storage and/or memory device. The storage and/or memory device can be one or more physical apparatuses used to store data or programs on a temporary or permanent basis. In some embodiments, the device can be volatile memory and require power to maintain stored information. In some embodiments, the device can be non-volatile memory and retain stored information when the digital processing device is not powered. In some embodiments, the non-volatile memory can include flash memory. In some embodiments, the non-volatile memory can include dynamic random-access memory (DRAM). In some embodiments, the non-volatile memory can include ferroelectric random access memory (FRAM). In some embodiments, the non-volatile memory can include phase-change random access memory (PRAM). In some embodiments, the device can be a storage device including, for example, CD-ROMs, DVDs, flash memory devices, magnetic disk drives, magnetic tapes drives, optical disk drives, and cloud computing-based storage. In some embodiments, the storage and/or memory device can be a combination of devices such as those disclosed herein. In some embodiments, the digital processing device can include a display to send visual information to a user. In some embodiments, the display can be a cathode ray tube (CRT). In some embodiments, the display can be a liquid crystal display (LCD). In some embodiments, the display can be a thin film transistor liquid crystal display (TFT-LCD). In some embodiments, the display can be an organic light emitting diode (OLED) display. In some embodiments, on OLED display can be a passive-matrix OLED (PMOLED) or active-matrix OLED (AMOLED) display. In some embodiments, the display can be a plasma display. In some embodiments, the display can be a video projector. In some embodiments, the display can be a combination of devices such as those disclosed herein.

In some embodiments, the digital processing device can include an input device to receive information from a user. In some embodiments, the input device can be a keyboard. In some embodiments, the input device can be a pointing device including, for example, a mouse, trackball, track pad, joystick, game controller, or stylus. In some embodiments, the input device can be a touch screen or a multi-touch screen. In some embodiments, the input device can be a microphone to capture voice or other sound input. In some embodiments, the input device can be a video camera to capture motion or visual input. In some embodiments, the input device can be a combination of devices such as those disclosed herein.

### E. Non-Transitory Computer-Readable Storage Medium

In some embodiments, the subject matter disclosed herein can include one or more non-transitory computer-readable storage media encoded with a program including instructions executable by the operating system of an optionally networked digital processing device. In some embodiments, a computer-readable storage medium can be a tangible component of a digital processing device. In some embodiments, a computer-readable storage medium can be optionally removable from a digital processing device. In some embodiments, a computer-readable storage medium can include, for example, CD-ROMs, DVDs, flash memory devices, solid state memory, magnetic disk drives, magnetic tape drives, optical disk drives, cloud computing systems and services, and the like. In some embodiments, the program and instructions can be permanently, substantially permanently, semi-permanently, or non-transitorily encoded on the media.

### F. Computer Systems

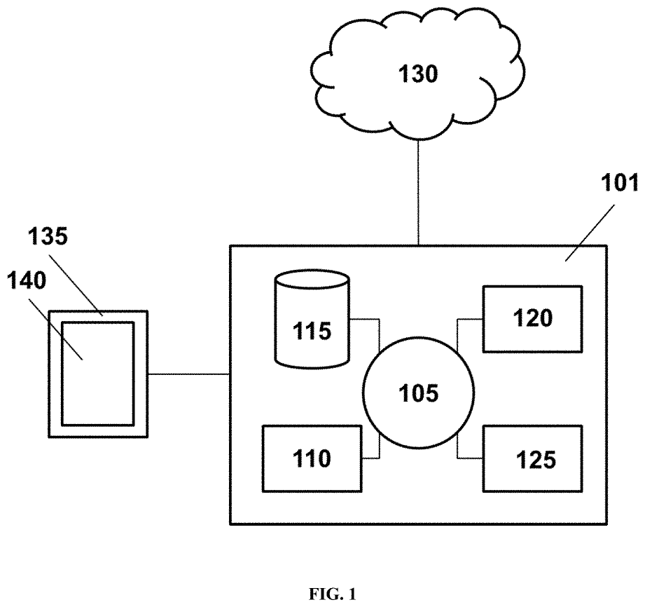

The present disclosure provides computer systems that are programmed to implement methods of the disclosure. FIG. 1 shows a computer system 101 that is programmed or otherwise configured to store, process, identify, or interpret patient data, biological data, biological sequences, or reference sequences (such as, e.g., map sequence reads obtained from sequencing nucleic acids to a reference nucleic acid sequence, separate sequence reads that do not map to a reference nucleic acid sequence to obtain presumed microbiome sequence reads, compare presumed microbiome sequence reads to a reference microbiome nucleic acid sequence to obtain actual microbiome sequence reads, apply a predictive model for classifying a subject to a disease or condition associated with the actual microbiome sequence reads of the subject, map sequences of gene expression products to microbiota, classify a biological sample as positive or negative for advanced adenoma or colorectal cancer using a trained algorithm to process the mapped sequences, output a report on a computer screen that identifies the biological sample as negative for the advanced adenoma or colorectal cancer, and apply a predictive model to actual microbiome sequence reads to classify a subject to detect the presence of cancer in the subject).

The computer system 101 can process various aspects of patient data, biological data, biological sequences, or reference sequences of the present disclosure (such as, e.g., mapping sequence reads obtained from sequencing nucleic acids to a reference nucleic acid sequence, separating sequence reads that do not map to a reference nucleic acid sequence to obtain presumed microbiome sequence reads, comparing presumed microbiome sequence reads to a reference microbiome nucleic acid sequence to obtain actual microbiome sequence reads, applying a predictive model for classifying a subject to a disease or condition associated with the actual microbiome sequence reads of the subject, mapping sequences of gene expression products to microbiota, classifying a biological sample as positive or negative for advanced adenoma or colorectal cancer using a trained algorithm to process the mapped sequences, outputting a report on a computer screen that identifies the biological sample as negative for the advanced adenoma or colorectal cancer, and applying a predictive model to actual microbiome sequence reads to classify a subject to detect the presence of cancer in the subject). The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some embodiments is a telecommunication and/or data network. The network 130 can include one or more computer servers, which can enable distributed computing, such as cloud computing. The network 130, in some embodiments with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some embodiments, the circuit is an application specific integrated circuit (ASIC).

The storage unit 115 can store files, such as drivers, libraries and saved programs. The storage unit 115 can store user data, e.g., user preferences and user programs. The computer system 101 in some embodiments can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some embodiments, the code can be retrieved from the storage unit 115 and stored on the memory 110 for ready access by the processor 105. In some embodiments, the electronic storage unit 115 can be precluded, and machine-executable instructions are stored on memory 110.

The code can be pre-compiled and configured for use with a machine having a processer adapted to execute the code or can be interpreted or compiled during runtime. The code can be supplied in a programming language that can be selected to enable the code to execute in a pre-compiled, interpreted, or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, a nucleic acid sequence, an enriched nucleic acid sample, an expression profile, and an analysis of an expression profile. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, probe a plurality of regulatory elements, sequence a nucleic acid sample, enrich a nucleic acid sample, determine an expression profile of a nucleic acid sample, analyze an expression profile of a nucleic acid sample, and archive or disseminate results of analysis of an expression profile. The algorithm can, for example, map sequence reads obtained from sequencing nucleic acids to a reference nucleic acid sequence, separate sequence reads that do not map to a reference nucleic acid sequence to obtain presumed microbiome sequence reads, compare presumed microbiome sequence reads to a reference microbiome nucleic acid sequence to obtain actual microbiome sequence reads, apply a predictive model for classifying a subject to a disease or condition associated with the actual microbiome sequence reads of the subject, map sequences of gene expression products to microbiota, classify a biological sample as positive or negative for advanced adenoma or colorectal cancer using a trained algorithm to process the mapped sequences, output a report on a computer screen that identifies the biological sample as negative for the advanced adenoma or colorectal cancer, and apply a predictive model to actual microbiome sequence reads to classify a subject to detect the presence of cancer in the subject.

In some embodiments, the subject matter disclosed herein can include at least one computer program or use of the same. A computer program can a sequence of instructions, executable in the digital processing device's CPU, GPU, or TPU, written to perform a specified task. Computer-readable instructions can be implemented as program modules, such as functions, objects, Application Programming Interfaces (APIs), data structures, and the like, that perform particular tasks or implement particular abstract data types. In light of the disclosure provided herein, it will be appreciated that a computer program can be written in various versions of various languages.

The functionality of the computer-readable instructions can be combined or distributed as desired in various environments. In some embodiments, a computer program can include one sequence of instructions. In some embodiments, a computer program can include a plurality of sequences of instructions. In some embodiments, a computer program can be provided from one location. In some embodiments, a computer program can be provided from a plurality of locations. In some embodiments, a computer program can include one or more software modules. In some embodiments, a computer program can include, in part or in whole, one or more web applications, one or more mobile applications, one or more standalone applications, one or more web browser plug-ins, extensions, add-ins, or add-ons, or combinations thereof.

In some embodiments, the computer processing can be a method of statistics, mathematics, biology, or any combination thereof. In some embodiments, the computer processing method includes a dimension reduction method including, for example, logistic regression, dimension reduction, principal component analysis, autoencoders, singular value decomposition, Fourier bases, singular value decomposition, wavelets, discriminant analysis, support vector machine, tree-based methods, random forest, gradient boost tree, logistic regression, matrix factorization, networkclustering, and neural network.

In some embodiments, the computer processing method is a supervised machine learning method including, for example, a regression, support vector machine, tree-based method, and network.

In some embodiments, the computer processing method is an unsupervised machine learning method including, for example, clustering, network, principal component analysis, and matrix factorization.

### G. Databases

In some embodiments, the subject matter disclosed herein can include one or more databases, or use of the same to store patient data, biological data, biological sequences, or reference sequences. Reference sequences can be derived from a database. In view of the disclosure provided herein, it will be appreciated that many databases can be suitable for storage and retrieval of the sequence information. In some embodiments, suitable databases can include, for example, relational databases, non-relational databases, object-oriented databases, object databases, entity-relationship model databases, associative databases, and XML databases. In some embodiments, a database can be internet-based. In some embodiments, a database can be web-based. In some embodiments, a database can be cloud computing-based. In some embodiments, a database can be based on one or more local computer storage devices.

In some embodiments, a database can represent a reference genome such as the Genome Reference Consortium GRCh38, Genome Reference Consortium GRCh37, NIH Human Microbiome Project (HMP v1 and v2), or Human Pan-Microbe Communities (HPMC), as well as National Center for Biotechnology Information (NCBI) EST or other sequence databases.

In one embodiment, the reference genome is selected from GrCH38, GrCH37, NA12878, or GM12878.

In various embodiments, the reference genome database is used for alignment and mapping steps of the methods disclosed herein.

### V. Diagnostic Methods

In one aspect, the disclosure provides a method of classifying an individual microbiome in a cell-free nucleic acid (cfNA) sample to identify a disease or condition of a subject

In one embodiment, the method comprises: (a) mapping a plurality of sequence reads obtained from sequencing a cell-free nucleic acid sample to a reference nucleic acid sequence; (b) separating sequence reads that do not map to a reference nucleic acid sequence, thereby providing presumed microbiome sequence reads; (c) comparing the presumed microbiome sequence reads to a reference microbiome nucleic acid sequence, wherein the presumed microbiome sequence reads that map to the reference microbiome nucleic acid sequence are actual microbiome sequence reads; and (d) applying a predictive model for classifying the subject to a disease or condition associated with the actual microbiome sequence reads of the subject.

### A. Subjects

In some embodiments, the present disclosure provides a system, method, or kit that includes or uses genomic material including, for example, cfDNA, from one or more subjects. In some embodiments, a subject is a biological entity containing expressed genetic materials. Examples of a biological entity include, but not limited to, a plant, animal, or microorganism, including, e.g., bacteria, viruses, fungi, and protozoa. In some embodiments, a subject includes tissues, cells, and progeny cells of a biological entity obtained in vivo or cultured in vitro.

In some embodiments, a subject is a mammal. In some embodiments, a subject is a human. In some embodiments, a human is a male or female. In additional embodiments, a human is from 1 day to about 1 year old, about 1 year old to about 3 years old, about 3 years old to about 12 years old, about 13 years old to about 19 years old, about 20 years old to about 40 years old, about 40 years old to about 65 years old, or over 65 years old.

In some embodiments, a subject is healthy or normal. In some embodiments, a subject is abnormal, or is diagnosed with, or suspected of being at a risk for, a disease. In some embodiments, a disease is a cancer, a disorder, a symptom, a condition, a syndrome, or any combination thereof.

### B. Diseases and Conditions

Conditions that can be inferred by the disclosed methods include, for example, cancer, gut-associated diseases, immune-mediated inflammatory diseases, neurological diseases, kidney diseases, prenatal diseases, and metabolic diseases.

Subjects with a disease or condition can be distinguished from subjects without that disease or condition by analyzing the taxonomic community composition of microbiota using sequencing results of cfDNA derived from the subjects.

The taxonomic community can include one or more of the following microbes: Abiotrophia, Abiotrophia defectiva, Acidobacteria, Acidovorax, Acinetobacter, Acetanaerobacteria, Actinobacteria, Actinomycetes, Aeromonas, Agrobacterium, Akkermansia, Alistipes, Allobaculum, Aquabacterium, Azonexus, Bacillaceae_1, Bacteroides, Bacteroidetes, Bifidobacterium, Bifidobacterium bifidum, Bryantella, Catonella, Carnobacteriaceae_1, Chryseobacterium, Chryseomonas, Cloacibacterium, Clostridiales, Clostridium, Clostridium difficile, Clostridium tetani, Coriobacterineae, Corynebacteria, Comamonas, Cyanobacteria, Dechloromonas, Delftia, Enterobacter, Enterobacteriaceae, Enterococcus faecalis, Escherichia coli, Erwinia, Exiguobacterium, Firmicutes, Flavimonas, Fusobacteria, Gp1, Gp2, Haemophilus influenza, Helicobacter, Hoidemania, Klebsiella, Klebsiella bacterium, Lachnospiraceae incertae sedis, Lactobacillus, Lactococcus, Leuconostoc, Methylobacterium, Micrococcineae, Mycobacteria, Neisseria, Neisseria meningitides, Novosphingobium, Oligotropha, Pantoea, Paiudibacter, Proteobacteria, Proteus, Pseudomonas, Pseudomonas aeruginosa, Pseudoxanthomonas, Raistonia, Rikeneia, Roseburia, Rubrobacterineae, Serratia, Shinella, Sphingobium, Spirochetes, Sporobacter, Staphylococcus, Staphylococcus aureus, Staphylococcus epidermidis, Staphylococcus mitis, Stenotrophomonas, Streptococcus mutans, Streptococcus pneumoniae, Streptococcus pyogenes, Streptococcus salivarius, Stenotrophomonas, Succinivibrio, Sutterella, Syntrophococcus, Turicibacter, Variovorax, Verrucomicrobia, and Weissella.

In one embodiment, the taxonomic community comprises one or more microbes selected from Propionibacterium spp., Candidatus Zinderia spp., Dasheen mosaic virus, Vicia cryptic virus, Comamonas spp., Caulobacter spp., Acinetobacter spp., Burkholdreia spp., Micrococcus spp., Candidatus Sulcia spp., Torque teno virus, Polaromonas spp., Pseudomonas spp., Acinetobacter spp., Cupriavidus spp., Dietzia spp., Neisseria spp., Propionibacterium spp., Stenotrophomonas spp., and combinations thereof.

In one embodiment, the taxonomic community comprises one or more microbes selected from Propionibacterium acnes, Candidatus Zinderia insecticola, Dasheen mosaic virus, Vicia cryptic virus, Comamonas spp., Caulobacter spp., Acinetobacter spp., Burkholdreia spp., Micrococcus luteus, Candidatus Sulcia muelleri, Torque teno virus, Polaromonas spp., Pseudomonas spp., Acinetobacter johnsonii, Cupriavidus spp., Dietzia spp., Neisseria spp., Propionibacterium granulosum, Stenotrophomonas maltophilia, and combinations thereof.

In one embodiment, the taxonomic community comprises one or more microbes selected from Propionibacterium acnes, Candidatus Zinderia insecticola, Dasheen mosaic virus, Vicia cryptic virus, and combinations thereof.

In one embodiment, the taxonomic community comprises one or more microbes selected from Propionibacterium acnes, Candidatus Zinderia insecticola, Dasheen mosaic virus, Vicia cryptic virus, Comamonas spp., Caulobacter spp., Acinetobacter spp., and combinations thereof.

In one embodiment, the taxonomic community comprises one or more microbes selected from Propionibacterium acnes, Candidatus Zinderia insecticola, Dasheen mosaic virus, Vicia cryptic virus, Comamonas spp., Caulobacter spp., Acinetobacter spp., Burkholdreia spp., Micrococcus luteus, Candidatus Sulcia muelleri, Torque teno virus, and combinations thereof.

In some embodiments, a biological condition can include a disease. In some embodiments, a biological condition can be a stage of a disease. In some embodiments, a biological condition can be a gradual change of a biological state. In some embodiments, a biological condition can be a treatment effect. In some embodiments, a biological condition can be a drug effect. In some embodiments, a biological condition can be a surgical effect. In some embodiments, a biological condition can be a biological state after a lifestyle modification. Non-limiting examples of lifestyle modifications include a diet change, a smoking change, and a sleeping pattern change.

In some embodiments, a biological condition is unknown. The analysis described herein can include machine learning to infer an unknown biological condition or to interpret the unknown biological condition.

In some embodiments, a method of the present disclosure can be used to diagnose a cancer. Non-limiting examples of cancers include adenoma (adenomatous polyps), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

Non-limiting examples of cancers that can be inferred by the disclosed methods include acute lymphoblastic leukemia (ALL), acute myeloid leukemia (AML), adrenocortical carcinoma, Kaposi sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloeptithelioma, pineal parenchymal tumor, breast cancer, bronchial tumor, Burkitt lymphoma, non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom macroglobulinemia, and Wilms tumor.

Non-limiting examples of gut-associated diseases that can be inferred by the disclosed methods include Crohn's disease, colitis, ulcerative colitis (UC), inflammatory bowel disease (IBD), irritable bowel syndrome (IBS), and celiac disease.

In various embodiments, the abnormal condition related to microbiota is a disease related to microbiota present in the animal body or the human body, wherein the microbiota is selected from the group consisting of microbiota found in the gastrointestinal tract, nasal passages, oral cavities, skin and the urogenital tract.

In various embodiments, the abnormal condition related to microbiota is a colorectal disease selected from the group consisting of colorectal cancer, advanced adenoma, ulcerative colitis, Crohn's disease, irritable bowel syndrome (IBS).

In one embodiment, the colorectal cancer is classified by stages such as stage 0, stage I, stage IIA, stage IIB, stage IIC, stage IIIA, stage IIIB, stage IIIC, stage IVA, stage IVB, or stage IVC.

In some embodiments, the disease is inflammatory bowel disease, colitis, ulcerative colitis, Crohn's disease, microscopic colitis, collagenous colitis, lymphocytic colitis, diversion colitis, Behçet's disease, and indeterminate colitis.

In various embodiments, an increase in relative abundance of cfDNA sequences derived from P. anaerobius, F. nucleatum, enterotoxigenic B. fragilis, or genotoxic E. coli contribute to classification of colorectal cancer.

In various embodiments, an increase in relative abundance of cfDNA sequences derived from H. pylori contributes to classification of gastric cancer.

In various embodiments, an increase in relative abundance of cfDNA sequences derived from H. hepaticus contributes to classification of liver cancer.

In various embodiments, an increase in relative abundance of cfDNA sequences derived from P. gingivalis contributes to classification of pancreatic cancer.

### C. Treatment Responsiveness

In various embodiments, presence and/or abundance of cfDNA sequences inform a classifier that stratifies a population of subjects according to responsiveness to a disease treatment.

In one embodiment, sequences derived from Akkermansia muciniphila inform a classifier that stratifies a population of subjects according to responsiveness to immunotherapy. In one embodiment, a detected level of sequences derived from Akkermansia muciniphila that is lower than normal level indicates a reduced response to immunotherapy.

In one embodiment, sequences derived from Clostridium species inform a classifier that stratifies a population of subjects according to rate of tumor growth. In one embodiment, a detected level of Clostridium species that is lower than normal levels indicates a reduced ability to control tumor growth and thus an increased rate of tumor growth.

In one embodiment, sequences derived from Bifidobacterium longum, Collinsella aerofaciens, and/or Enterococcus faecium inform a classifier that stratifies a population of subjects according to response to anti-PD-1-based immunotherapy. In one embodiment, Bifidobacterium longum, Collinsella aerofaciens, and Enterococcus faecium having a higher relative abundance than normal indicates an increased response to anti-PD-1-based immunotherapy.

In one embodiment, sequences derived from Ruminococcaceae family inform a classifier that stratifies a population of subjects according to response to PD-1 blockade. In one embodiment, higher alpha diversity (P<0.01) and relative abundance of bacteria of the Ruminococcaceae family (P<0.01) indicates melanoma patients responding to PD-1 blockade.

In one embodiment, sequences derived from Fusobacterium nucleatum inform a classifier that stratifies a population of subjects according to recurrence of colorectal cancer following chemotherapy treatment. In one embodiment, Fusobacterium nucleatum at higher relative abundance than normal indicates recurrence of colorectal cancer following chemotherapy treatment.

### D. Risk States

In some embodiments, the present disclosure provides a system, method, or kit that includes a first sample and a second sample collected from a subject that differ by risk for developing a biological condition. In some embodiments, the system, method, or kit disclosed herein can include evaluating or predicting a risk state.

In some embodiments, a risk state can include the risk for developing a disease state. In some embodiments, a risk state can be a stage of a disease. In some embodiments, the risk state can be an age-associated disease. In some embodiments, a risk state can include one or more aspects associated with aging. In some embodiments, a risk state can be a state in aging. In some embodiments, a risk state can be a treatment effect, side effect, or non-intended impact of medical treatment. In some embodiments, a risk state can be a surgical outcome. In some embodiments, a risk effect can be a biological state that can occur after a lifestyle modification. Non-limiting examples of lifestyle modifications include a diet change, a smoking change, and a sleeping pattern change.

In some embodiments, a risk state is unknown. The present disclosure provides a system, method, or kit that can include machine learning to infer an unknown risk state or to interpret the unknown risk state.

Vi. Kits and Systems

In some embodiments, the present disclosure provides a system, method, or kit that can include a first and a second sample collected from a same subject at different times (e.g., before and after entering a disease state). In some embodiments, the system, method, or kit disclosed herein can include evaluating or predicting a disease or condition. In some embodiments, the system, media, method, or kit disclosed herein can include evaluating or predicting a state of a disease or condition. The state or condition can be past, present, or future.

The present disclosure provides kits for identifying or monitoring a disease or disorder (e.g., cancer) of a subject. A kit may comprise probes for identifying a quantitative measure (e.g., indicative of a presence, absence, or relative amount) of sequences at each of a panel of cancer-associated genomic loci or microbiome-associated genomic loci in a sample of the subject. A quantitative measure (e.g., indicative of a presence, absence, or relative amount) of sequences at each of a panel of cancer-associated genomic loci or microbiome-associated genomic loci in the sample may be indicative of the disease or disorder (e.g., cancer) of the subject. The probes may be selective for the sequences at the panel of cancer-associated genomic loci or microbiome-associated genomic loci in the sample. A kit may comprise instructions for using the probes to process the sample to generate datasets indicative of a quantitative measure (e.g., indicative of a presence, absence, or relative amount) of sequences at each of the panel of cancer-associated genomic loci or microbiome-associated genomic loci in a sample of the subject.

The probes in the kit may be selective for the sequences at the panel of cancer-associated genomic loci or microbiome-associated genomic loci in the sample. The probes in the kit may be configured to selectively enrich nucleic acid (e.g., RNA or DNA) molecules corresponding to the panel of cancer-associated genomic loci or microbiome-associated genomic loci. The probes in the kit may be nucleic acid primers. The probes in the kit may have sequence complementarity with nucleic acid sequences from one or more of the panel of cancer-associated genomic loci or microbiome-associated genomic loci or genomic regions. The panel of cancer-associated genomic loci or microbiome-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct panel of cancer-associated genomic loci or microbiome-associated genomic loci or genomic regions.

The instructions in the kit may comprise instructions to assay the sample using the probes that are selective for the sequences at the panel of cancer-associated genomic loci or microbiome-associated genomic loci in the cell-free biological sample. These probes may be nucleic acid molecules (e.g., RNA or DNA) having sequence complementarity with nucleic acid sequences (e.g., RNA or DNA) from one or more of the plurality of panel of cancer-associated genomic loci or microbiome-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of cancer-associated genomic loci or microbiome-associated genomic loci in the sample. A quantitative measure (e.g., indicative of a presence, absence, or relative amount) of sequences at each of a panel of cancer-associated genomic loci or microbiome-associated genomic loci in the sample may be indicative of a disease or disorder (e.g., cancer).

The instructions in the kit may comprise instructions to measure and interpret assay readouts, which may be quantified at one or more of the panel of cancer-associated genomic loci to generate the datasets indicative of a quantitative measure (e.g., indicative of a presence, absence, or relative amount) of sequences at each of the panel of cancer-associated genomic loci or microbiome-associated genomic loci in the sample. For example, quantification of array hybridization or polymerase chain reaction (PCR) corresponding to the panel of cancer-associated genomic loci or microbiome-associated genomic loci may generate the datasets indicative of a quantitative measure (e.g., indicative of a presence, absence, or relative amount) of sequences at each of the panel of cancer-associated genomic loci or microbiome-associated genomic loci in the sample. Assay readouts may comprise quantitative PCR (qPCR) values, digital PCR (dPCR) values, digital droplet PCR (ddPCR) values, fluorescence values, etc., or normalized values thereof.

### EXAMPLES

### Example 1

Methods of using principal component analysis to detect advanced adenoma in cfDNA samples in a population.

A principal component analysis (PCA) was used to assess the performance of the disclosed methods of detecting advanced adenoma in a population (e.g., versus colorectal carcinoma and healthy samples).

To generate a set of training data, cell-free DNA samples were obtained from four groups of subjects: a first group of subjects with advanced adenoma (AA), a second group of subjects with colorectal carcinoma (CRC), a third group of healthy donors (HD), and a fourth group of subjects with inflammatory bowel disease (IBD). Healthy donor samples were obtained from healthy subjects, or subjects who do not have or have not been diagnosed with any of the above indications. The cell-free DNA samples were processed through plasma isolation, cfDNA extraction, sequencing library preparation, and deep whole genome sequencing to obtain data comprising nucleic acid sequences.

The nucleic acid sequences were mapped to a human reference genome GrCH38, and the mapped sequences were removed from analysis. The unmapped sequences, which are of presumptive microbiome content in the sample, were isolated for further analysis. The BWA alignment tool was used to align the unmapped sequence reads (e.g., the taxonomic microbiota community composition) to an all-microbiome reference genome. (215 30×WGS samples, ˜50 samples each). The alignment was analyzed by disease, batch ID, and date to rule out any batch effects, which may confound the analysis.

The taxonomic microbiota community composition of the cfDNA samples were identified using Metagenomic Phylogenetic Analysis (for example, MetaPhlAn2 or MetaPhlAn v2.0) to map all unmapped sequence reads from deep whole genome sequencing onto taxonomic-specific genetic markers that were summarized from the Human Microbiome Project. The taxonomic community composition of microbiota was calculated by estimating the normalized number of sequence reads that mapped to the taxonomic-specific genetic markers.

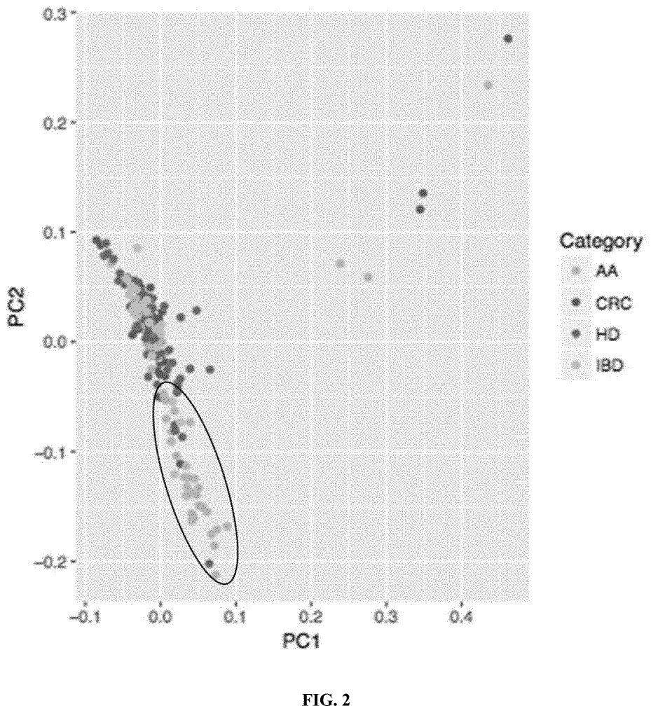

A feature matrix was generated from normalized number of sequence reads for each sample from each level of taxonomic (kingdom, phylum, class, order, family, genus, and species) or the relative abundance of taxonomic community composition of the microbiota. A PCA plot of the feature matrix was generated, which showed that the AA samples were largely separated from the other sample populations (CRC, HD, and IBD), as shown in FIG. 2 (circled).

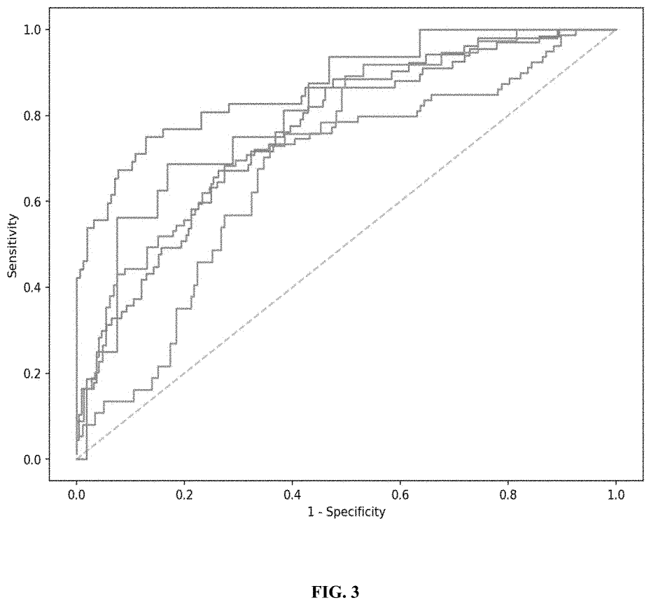

A receiver operating characteristic (ROC) curve was used to assess the performance of identifying AA samples using the disclosed method. Machine learning methods, such as random forest, logistic regression, and multilayer perceptron (MLP), were applied to the training data to generate a classifier capable of distinguishing AA subjects from healthy subjects with 54% sensitivity and 85% specificity (as shown in FIG. 3 and TABLE 2) of identifying AA samples. Sensitivity and specificity were much higher than those achieved using other non-invasive AA screening methods, as described in TABLE 2.

TABLE 2
The performance metrics for the classification
of AA and healthy samples

| | Sensitivity at | Standard | | Standard |
|---|---|---|---|---|
| Training Set | 85% Specificity | Deviation | AUC | Deviation |
| AA vs. | 0.54 | 0.163 | 0.77 | 0.056 |
| healthy controls | | | | |

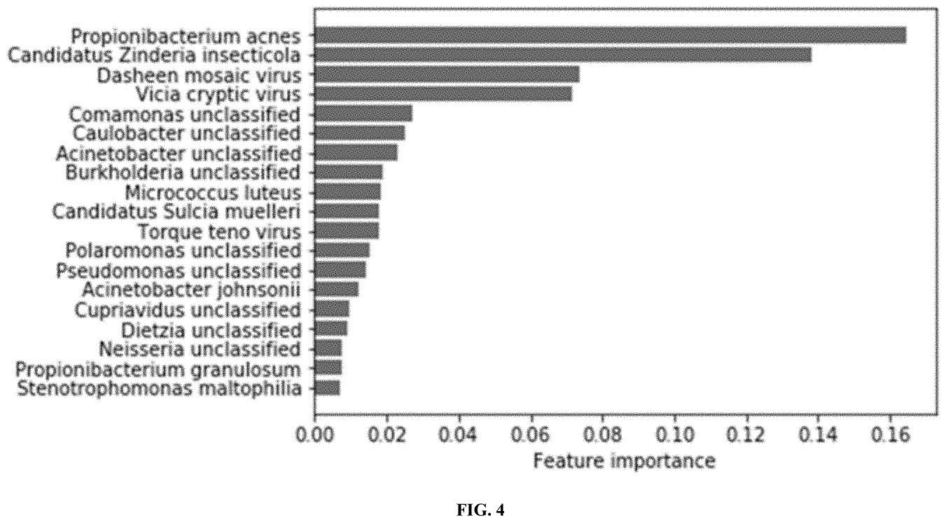

Performing a characterization process can include determining feature relevance scores and/or other suitable metrics associated with feature importance (e.g., through applying random forest techniques); and using the feature relevance scores and/or other suitable metrics, along with supplemental data (e.g., prior biological knowledge informative of the microbiome features, such as with a third microbiome characterization modules, Analytical Module F, etc.) to obtain sample-level quantification of microbiome functional features (e.g., using any suitable software tools). Biomarker weight optimization included calculating feature importance using random forest regression, in which abundant biomarkers are assigned higher importance for distinguishing between samples from AA and healthy subjects. The results of feature importance analysis are shown as a feature importance rank plot for the classification of AA vs. healthy samples in FIG. 4.

Microbiome taxa principal components can be used as predictors (e.g., predictor variables) of the advanced adenoma disease conditions with two labels: healthy or AA, where a machine learning classifier (e.g., random forest classifier) can be generated from the training data for determining feature relevance scores and/or other feature importance metric (e.g., for determining the most important microbiome sub-system's principal component predictor, etc.). In the specific example, as shown in TABLE 3 and FIG. 4, feature importance metrics identified a ranking of relevance for the different microbiome sequences identified in the sample, where Propionibacterium acnes and Candidatus Zinderia insectola are identified as the two most relevant features for identifying AA.

TABLE 3
Microbiome Elements Relevant to Advanced Adenoma Classification
Taxonomic Name

Propionibacterium spp.
Candidatus Zinderia spp.
Dasheen mosaic virus
Vicia cryptic virus
Comamonas spp.
Caulobacter spp.
Acinetobacter spp.
Burkholdreia spp.
Micrococcus spp.
Candidatus Sulcia spp.
Torque teno virus
Polaromonas spp.
Pseudomonas spp.
Acinetobacter spp.
Cupriavidus spp.
Dietzia spp.
Neisseria spp.
Propionibacterium spp.
Stenotrophomonas spp.

### Example 2

Methods to detect colorectal cancer in cfDNA samples in a population.

A principal component analysis (PCA) is used to assess the performance of the disclosed methods of detecting colorectal cancer (CRC) in a population (e.g., vs. healthy samples).

To generate a set of training data, cell-free DNA samples are obtained from subjects having colorectal cancer. Healthy donor (HD) cell-free DNA samples are obtained from healthy subjects, or subjects who do not have or have not been diagnosed with colorectal cancer. The cell-free DNA samples are processed through plasma isolation, cfDNA extraction, sequencing library preparation, and deep whole genome sequencing to obtain data comprising nucleic acid sequences.

The nucleic acid sequences are mapped to a human reference genome GrCH38, and the mapped sequences are removed from analysis. The unmapped sequences, which are of presumptive microbiome content in the sample, are isolated for further analysis. The BWA alignment tool is used to align the unmapped sequence reads (e.g., the taxonomic microbiota community composition) to an all-microbiome reference genome. (215 30×WGS samples, ˜50 samples each). The alignment is analyzed by disease, batch ID, and date to rule out any batch effects, which may confound the analysis.

The taxonomic microbiota community composition of the cfDNA samples are identified using Metagenomic Phylogenetic Analysis (for example, MetaPhlAn2 or MetaPhlAn v2.0) to map all unmapped sequence reads from deep whole genome sequencing onto taxonomic-specific genetic markers that were summarized from the Human Microbiome Project. The taxonomic community composition of microbiota is calculated by estimating the normalized number of sequence reads that mapped to the taxonomic-specific genetic markers.

A feature matrix is generated from normalized number of sequence reads for each sample from each level of taxonomic (kingdom, phylum, class, order, family, genus, and species) or the relative abundance of taxonomic community composition of the microbiota. A PCA plot of the feature matrix is generated to show colorectal cancer samples that are separated from the other healthy donor sample populations.

The predictive model and classifier are used to classify cfDNA samples isolated from subjects suspected of having colorectal cancer. A receiver operating characteristic (ROC) curve is used to assess the performance of identifying CRC samples using the disclosed method. Machine learning methods, such as random forest, logistic regression, and multilayer perceptron (MLP), are applied to the training data to generate a classifier capable of distinguishing CRC subjects from healthy subjects with high sensitivity and specificity of identifying CRC samples.

Performing a characterization process includes determining feature relevance scores and/or other suitable metrics associated with feature importance (e.g., through applying random forest techniques); and using the feature relevance scores and/or other suitable metrics, along with supplemental data (e.g., prior biological knowledge informative of the microbiome features, such as with a third microbiome characterization modules, Analytical Module F, etc.) to obtain sample-level quantification of microbiome functional features (e.g., using any suitable software tools). Biomarker weight optimization includes calculating feature importance using random forest regression, in which abundant biomarkers are assigned higher importance for distinguishing between samples from CRC and healthy subjects. The results of feature importance analysis are shown using a feature importance rank plot for the classification of CRC vs. healthy samples.

Microbiome taxa principal components are used as predictors (e.g., predictor variables) of the CRC disease conditions with two labels: healthy or CRC, where a machine learning classifier (e.g., random forest classifier) is generated from the training data for determining feature relevance scores and/or other feature importance metric (e.g., for determining the most important microbiome sub-system's principal component predictor, etc.). Feature importance metrics are used to identify a ranking of relevance for the different microbiome sequences identified in the sample, to identify a number of most relevant subset of features from among the set of features for identifying CRC.

### Example 3

Methods to detect liver cancer in cfDNA samples in a population. A principal component analysis (PCA) is used to assess the performance of the disclosed methods of detecting liver cancer in a population (e.g., vs. healthy samples).

To generate a set of training data, cell-free DNA samples are obtained from subjects having liver cancer. Healthy donor (HD) cell-free DNA samples are obtained from healthy subjects, or subjects who do not have or have not been diagnosed with liver cancer. The cell-free DNA samples are processed through plasma isolation, cfDNA extraction, sequencing library preparation, and deep whole genome sequencing to obtain data comprising nucleic acid sequences.

The nucleic acid sequences are mapped to a human reference genome GrCH38, and the mapped sequences are removed from analysis. The unmapped sequences, which are of presumptive microbiome content in the sample, are isolated for further analysis. The BWA alignment tool is used to align the unmapped sequence reads (e.g., the taxonomic microbiota community composition) to an all-microbiome reference genome. (215 30×WGS samples, ˜50 samples each). The alignment is analyzed by disease, batch ID, and date to rule out any batch effects, which may confound the analysis.

The taxonomic microbiota community composition of the cfDNA samples are identified using Metagenomic Phylogenetic Analysis (for example, MetaPhlAn2 or MetaPhlAn v2.0) to map all unmapped sequence reads from deep whole genome sequencing onto taxonomic-specific genetic markers that were summarized from the Human Microbiome Project. The taxonomic community composition of microbiota is calculated by estimating the normalized number of sequence reads that mapped to the taxonomic-specific genetic markers.

A feature matrix is generated from normalized number of sequence reads for each sample from each level of taxonomic (kingdom, phylum, class, order, family, genus, and species) or the relative abundance of taxonomic community composition of the microbiota. A PCA plot of the feature matrix is generated to show liver cancer samples that are separated from the other healthy donor sample populations.

The predictive model and classifier are used to classify cfDNA samples isolated from subjects suspected of having liver cancer. A receiver operating characteristic (ROC) curve is used to assess the performance of identifying liver cancer samples using the disclosed method. Machine learning methods, such as random forest, logistic regression, and multilayer perceptron (MLP), are applied to the training data to generate a classifier capable of distinguishing liver cancer subjects from healthy subjects with high sensitivity and specificity of identifying liver cancer samples.

Performing a characterization process includes determining feature relevance scores and/or other suitable metrics associated with feature importance (e.g., through applying random forest techniques); and using the feature relevance scores and/or other suitable metrics, along with supplemental data (e.g., prior biological knowledge informative of the microbiome features, such as with a third microbiome characterization modules, Analytical Module F, etc.) to obtain sample-level quantification of microbiome functional features (e.g., using any suitable software tools). Biomarker weight optimization includes calculating feature importance using random forest regression, in which abundant biomarkers are assigned higher importance for distinguishing between samples from liver cancer and healthy subjects. The results of feature importance analysis are shown using a feature importance rank plot for the classification of liver cancer vs. healthy samples.

Microbiome taxa principal components can be used as predictors (e.g., predictor variables) of the liver cancer disease conditions with two labels: healthy or liver cancer, where a machine learning classifier (e.g., random forest classifier) can be generated from the training data for determining feature relevance scores and/or other feature importance metric (e.g., for determining the most important microbiome sub-system's principal component predictor, etc.). Feature importance metrics are used to identify a ranking of relevance for the different microbiome sequences identified in the sample, to identify a number of most relevant subset of features from among the set of features for identifying liver cancer.

### Example 4

Methods to detect breast cancer in cfDNA samples in a population.

A principal component analysis (PCA) is used to assess the performance of the disclosed methods of detecting breast cancer in a population (e.g., vs. healthy samples).

To generate a set of training data, cell-free DNA samples are obtained from subjects having breast cancer. Healthy donor (HD) cell-free DNA samples are obtained from healthy subjects, or subjects who do not have or have not been diagnosed with breast cancer. The cell-free DNA samples are processed through plasma isolation, cfDNA extraction, sequencing library preparation, and deep whole genome sequencing to obtain data comprising nucleic acid sequences.

The nucleic acid sequences are mapped to a human reference genome GrCH38, and the mapped sequences are removed from analysis. The unmapped sequences, which are of presumptive microbiome content in the sample, are isolated for further analysis. The BWA alignment tool is used to align the unmapped sequence reads (e.g., the taxonomic microbiota community composition) to an all-microbiome reference genome. (215 30×WGS samples, ˜50 samples each). The alignment is analyzed by disease, batch ID, and date to rule out any batch effects, which may confound the analysis.

The taxonomic microbiota community composition of the cfDNA samples are identified using Metagenomic Phylogenetic Analysis (for example, MetaPhlAn2 or MetaPhlAn v2.0) to map all unmapped sequence reads from deep whole genome sequencing onto taxonomic-specific genetic markers that were summarized from the Human Microbiome Project. The taxonomic community composition of microbiota is calculated by estimating the normalized number of sequence reads that mapped to the taxonomic-specific genetic markers.

A feature matrix is generated from normalized number of sequence reads for each sample from each level of taxonomic (kingdom, phylum, class, order, family, genus, and species) or the relative abundance of taxonomic community composition of the microbiota. A PCA plot of the feature matrix is generated to show breast cancer samples that are separated from the other healthy donor sample populations.

The predictive model and classifier are used to classify cfDNA samples isolated from subjects suspected of having breast cancer. A receiver operating characteristic (ROC) curve is used to assess the performance of identifying breast cancer samples using the disclosed method. Machine learning methods, such as random forest, logistic regression, and multilayer perceptron (MLP), are applied to the training data to generate a classifier capable of distinguishing breast cancer subjects from healthy subjects with high sensitivity and specificity of identifying breast cancer samples.

Performing a characterization process includes determining feature relevance scores and/or other suitable metrics associated with feature importance (e.g., through applying random forest techniques); and using the feature relevance scores and/or other suitable metrics, along with supplemental data (e.g., prior biological knowledge informative of the microbiome features, such as with a third microbiome characterization modules, Analytical Module F, etc.) to obtain sample-level quantification of microbiome functional features (e.g., using any suitable software tools). Biomarker weight optimization includes calculating feature importance using random forest regression, in which abundant biomarkers are assigned higher importance for distinguishing between samples from breast cancer and healthy subjects. The results of feature importance analysis are shown using a feature importance rank plot for the classification of breast cancer vs. healthy samples.

Microbiome taxa principal components are used as predictors (e.g., predictor variables) of the breast cancer disease conditions with two labels: healthy or breast cancer, where a machine learning classifier (e.g., random forest classifier) are generated from the training data for determining feature relevance scores and/or other feature importance metric (e.g., for determining the most important microbiome sub-system's principal component predictor, etc.). Feature importance metrics are used to identify a ranking of relevance for the different microbiome sequences identified in the sample, to identify a number of most relevant subset of features from among the set of features for identifying breast cancer.

### Example 5

Methods to detect pancreatic cancer in cfDNA samples in a population.

A principal component analysis (PCA) is used to assess the performance of the disclosed methods of detecting pancreatic cancer in a population (e.g., vs. healthy samples). To generate a set of training data, cell-free DNA samples are obtained from subjects having pancreatic cancer. Healthy donor (HD) cell-free DNA samples are obtained from healthy subjects, or subjects who do not have or have not been diagnosed with pancreatic cancer. The cell-free DNA samples are processed through plasma isolation, cfDNA extraction, sequencing library preparation, and deep whole genome sequencing to obtain data comprising nucleic acid sequences.

The nucleic acid sequences are mapped to a human reference genome GrCH38, and the mapped sequences are removed from analysis. The unmapped sequences, which are of presumptive microbiome content in the sample, are isolated for further analysis. The BWA alignment tool is used to align the unmapped sequence reads (e.g., the taxonomic microbiota community composition) to an all-microbiome reference genome. (215 30×WGS samples, ˜50 samples each). The alignment is analyzed by disease, batch ID, and date to rule out any batch effects, which may confound the analysis.

The taxonomic microbiota community composition of the cfDNA samples are identified using Metagenomic Phylogenetic Analysis (for example, MetaPhlAn2 or MetaPhlAn v2.0) to map all unmapped sequence reads from deep whole genome sequencing onto taxonomic-specific genetic markers that were summarized from the Human Microbiome Project. The taxonomic community composition of microbiota is calculated by estimating the normalized number of sequence reads that mapped to the taxonomic-specific genetic markers.

A feature matrix is generated from normalized number of sequence reads for each sample from each level of taxonomic (kingdom, phylum, class, order, family, genus, and species) or the relative abundance of taxonomic community composition of the microbiota. A PCA plot of the feature matrix is generated to show pancreatic cancer samples that are separated from the other healthy donor sample populations.

The predictive model and classifier are used to classify cfDNA samples isolated from subjects suspected of having pancreatic cancer. A receiver operating characteristic (ROC) curve is used to assess the performance of identifying pancreatic cancer samples using the disclosed method. Machine learning methods, such as random forest, logistic regression, and multilayer perceptron (MLP), are applied to the training data to generate a classifier capable of distinguishing pancreatic cancer subjects from healthy subjects with high sensitivity and specificity of identifying pancreatic cancer samples.

Performing a characterization process includes determining feature relevance scores and/or other suitable metrics associated with feature importance (e.g., through applying random forest techniques); and using the feature relevance scores and/or other suitable metrics, along with supplemental data (e.g., prior biological knowledge informative of the microbiome features, such as with a third microbiome characterization modules, Analytical Module F, etc.) to obtain sample-level quantification of microbiome functional features (e.g., using any suitable software tools). Biomarker weight optimization includes calculating feature importance using random forest regression, in which abundant biomarkers are assigned higher importance for distinguishing between samples from pancreatic cancer and healthy subjects. The results of feature importance analysis are shown using a feature importance rank plot for the classification of pancreatic cancer vs. healthy samples.

Microbiome taxa principal components are used as predictors (e.g., predictor variables) of the pancreatic cancer disease conditions with two labels: healthy or pancreatic cancer, where a machine learning classifier (e.g., random forest classifier) are generated from the training data for determining feature relevance scores and/or other feature importance metric (e.g., for determining the most important microbiome sub-system's principal component predictor, etc.). Feature importance metrics are used to identify a ranking of relevance for the different microbiome sequences identified in the sample, to identify a number of most relevant subset of features from among the set of features for identifying pancreatic cancer.

### Example 6

Methods to stratify anti-PD1 treatment responders vs. non-responders in cfDNA samples in a population.

A principal component analysis (PCA) is used to assess the performance of the disclosed methods of stratifying anti-PD1 cancer treatment responders vs. non-responders in a population.

To generate a set of training data, cell-free DNA samples are obtained from subjects being treated for cancer with an anti-PD1 therapy (such as nivolumab, pembrolizumab, pidilizumab, or atezolizumab). For each subjects treated with an anti-PD1 therapy, the history or responsiveness or resistance to anti-PD1 therapy is also noted, to place the subjects into a group of responders or a group of non-responders. The cell-free DNA samples are processed through plasma isolation, cfDNA extraction, sequencing library preparation, and deep whole genome sequencing to obtain data comprising nucleic acid sequences.

The nucleic acid sequences are mapped to a human reference genome GrCH38, and the mapped sequences are removed from analysis. The unmapped sequences, which are of presumptive microbiome content in the sample, are isolated for further analysis. The BWA alignment tool is used to align the unmapped sequence reads (e.g., the taxonomic microbiota community composition) to an all-microbiome reference genome. (215 30×WGS samples, ˜50 samples each). The alignment is analyzed by disease, batch ID, and date to rule out any batch effects, which may confound the analysis.

The taxonomic microbiota community composition of the cfDNA samples are identified using Metagenomic Phylogenetic Analysis (for example, MetaPhlAn2 or MetaPhlAn v2.0) to map all unmapped sequence reads from deep whole genome sequencing onto taxonomic-specific genetic markers that were summarized from the Human Microbiome Project. The taxonomic community composition of microbiota is calculated by estimating the normalized number of sequence reads that mapped to the taxonomic-specific genetic markers.

A feature matrix is generated from normalized number of sequence reads for each sample from each level of taxonomic (kingdom, phylum, class, order, family, genus, and species) or the relative abundance of taxonomic community composition of the microbiota. A PCA plot of the feature matrix is generated to show anti-PD1 treatment responsiveness status of samples (e.g., responders) that are separated from non-responders.

The predictive model and classifier are used to classify cfDNA samples isolated from subjects being treated or to-be-treated with anti-PD1 therapy to stratify the population into responders and non-responders. A receiver operating characteristic (ROC) curve is used to assess the performance of distinguishing responder samples and non-responder samples using the disclosed method. Machine learning methods, such as random forest, logistic regression, and multilayer perceptron (MLP), are applied to the training data to generate a classifier capable of distinguishing responder subjects from non-responder subjects with high sensitivity and specificity of identifying responder samples.

Performing a characterization process includes determining feature relevance scores and/or other suitable metrics associated with feature importance (e.g., through applying random forest techniques); and using the feature relevance scores and/or other suitable metrics, along with supplemental data (e.g., prior biological knowledge informative of the microbiome features, such as with a third microbiome characterization modules, Analytical Module F, etc.) to obtain sample-level quantification of microbiome functional features (e.g., using any suitable software tools). Biomarker weight optimization includes calculating feature importance using random forest regression, in which abundant biomarkers are assigned higher importance for distinguishing between samples from responder subjects and non-responder subjects. The results of feature importance analysis are shown using a feature importance rank plot for the classification of responder vs. non-responder samples.

Microbiome taxa principal components are used as predictors (e.g., predictor variables) of the responders vs. non-responders with two labels: responder or non-responder, where a machine learning classifier (e.g., random forest classifier) are generated from the training data for determining feature relevance scores and/or other feature importance metric (e.g., for determining the most important microbiome sub-system's principal component predictor, etc.). Feature importance metrics are used to identify a ranking of relevance for the different microbiome sequences identified in the sample, to identify a number of most relevant subset of features from among the set of features for distinguishing responders vs. non-responders.

### Example 7

Methods to stratify anti-CTLA4 treatment responders vs. non-responders in cfDNA samples in a population

A principal component analysis (PCA) is used to assess the performance of the disclosed methods of stratifying anti-CTLA4 cancer treatment responders vs. non-responders in a population.

To generate a set of training data, cell-free DNA samples are obtained from subjects being treated for cancer with an anti-CTLA4 therapy (such as ipilimumab or tremelimumab). For each subject treated with an anti-CTLA4 therapy, the history or responsiveness or resistance to anti-CTLA4 therapy is also noted, to place the subject into a group of responders or a group of non-responders. The cell-free DNA samples are processed through plasma isolation, cfDNA extraction, sequencing library preparation, and deep whole genome sequencing to obtain data comprising nucleic acid sequences.

The nucleic acid sequences are mapped to a human reference genome GrCH38, and the mapped sequences are removed from analysis. The unmapped sequences, which are of presumptive microbiome content in the sample, are isolated for further analysis. The BWA alignment tool is used to align the unmapped sequence reads (e.g., the taxonomic microbiota community composition) to an all-microbiome reference genome. (215 30×WGS samples, ˜50 samples each). The alignment is analyzed by disease, batch ID, and date to rule out any batch effects, which may confound the analysis.

The taxonomic microbiota community composition of the cfDNA samples are identified using Metagenomic Phylogenetic Analysis (for example, MetaPhlAn2 or MetaPhlAn v2.0) to map all unmapped sequence reads from deep whole genome sequencing onto taxonomic-specific genetic markers that were summarized from the Human Microbiome Project. The taxonomic community composition of microbiota is calculated by estimating the normalized number of sequence reads that mapped to the taxonomic-specific genetic markers.

A feature matrix is generated from normalized number of sequence reads for each sample from each level of taxonomic (kingdom, phylum, class, order, family, genus, and species) or the relative abundance of taxonomic community composition of the microbiota. A PCA plot of the feature matrix is generated to show anti-CTLA4 therapy responsiveness status of samples (e.g., responders) that are separated from non-responders.

The predictive model and classifier are used to classify cfDNA samples isolated from subjects being treated or to-be-treated with anti-CTLA4 therapy to stratify the population into responders and non-responders. A receiver operating characteristic (ROC) curve is used to assess the performance of distinguishing responder samples and non-responder samples using the disclosed method. Machine learning methods, such as random forest, logistic regression, and multilayer perceptron (MLP), are applied to the training data to generate a classifier capable of distinguishing responder subjects from non-responder subjects with high sensitivity and specificity of identifying responder samples.

Performing a characterization process includes determining feature relevance scores and/or other suitable metrics associated with feature importance (e.g., through applying random forest techniques); and using the feature relevance scores and/or other suitable metrics, along with supplemental data (e.g., prior biological knowledge informative of the microbiome features, such as with a third microbiome characterization modules, Analytical Module F, etc.) to obtain sample-level quantification of microbiome functional features (e.g., using any suitable software tools). Biomarker weight optimization includes calculating feature importance using random forest regression, in which abundant biomarkers are assigned higher importance for distinguishing between samples from responder subjects and non-responder subjects. The results of feature importance analysis are shown using a feature importance rank plot for the classification of responder vs. non-responder samples.

Microbiome taxa principal components are used as predictors (e.g., predictor variables) of the responders vs. non-responders with two labels: responder or non-responder, where a machine learning classifier (e.g., random forest classifier) are generated from the training data for determining feature relevance scores and/or other feature importance metric (e.g., for determining the most important microbiome sub-system's principal component predictor, etc.). Feature importance metrics are used to identify a ranking of relevance for the different microbiome sequences identified in the sample, to identify a number of most relevant subset of features from among the set of features for distinguishing responders vs. non-responders.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing an invention of the disclosure. It is therefore contemplated that the invention shall also cover any such alternatives, modifications, variations or equivalents. It is intended that the following claims define the scope of the invention and that methods and structures within the scope of these claims and their equivalents be covered thereby.

1-44. (canceled)

45. A method for creating a data analysis module for classification of a gut-associated disease or cancer in a subject comprising:
(a) providing a cell-free nucleic acid sample obtained from plasma of said subject;
(b) sequencing said cell-free nucleic acid sample to provide a plurality of sequence reads;
(c) mapping said plurality of sequence reads to a human reference nucleic acid sequence;
(d) separating sequence reads that do not map to said human reference nucleic acid sequence, thereby providing presumed microbiome sequence reads;
(e) comparing said presumed microbiome sequence reads to a reference microbiome nucleic acid sequence, wherein said presumed microbiome sequence reads that map to said reference microbiome nucleic acid sequence are actual microbiome sequence reads; and
(f) using said actual microbiome sequence reads to create said data analysis module to classify said gut-associated disease or cancer of said subject.

46. The method of claim 45, wherein said data analysis module comprises methods selected from logistic regression, dimension reduction, principal component analysis, autoencoders, singular value decomposition, Fourier bases, singular value decomposition, wavelets, discriminant analysis, support vector machine, tree-based methods, random forest, gradient boost tree, logistic regression, matrix factorization, network clustering, and neural network.

47. The method of claim 45, wherein said data analysis module comprises principal component analysis.

48. The method of claim 45, wherein said data analysis module comprises unsupervised machine learning.

49. The method of claim 45, wherein said data analysis module comprises identifying a sequence variant inferred from sequence data based on probabilistic modeling, statistical modeling, mechanistic modeling, network modeling, or statistical inferences.

50. The method of claim 45, wherein said actual microbiome sequence reads represent a plurality of species of microbiota selected from Propionibacterium acnes, Candidatus Zonderia insecticola, Dasheen mosaic virus, Vicia cryptic virus, Comamonas spp., Caulobacter spp., Acinetobacter spp., Burkholdreia spp., Micrococcus luteus, Candidatus Sulcia muelleri, Torque teno virus, Polaromonas spp., Pseudomonas spp., Acinetobacter johnsonii, Cupriavidus spp., Dietzia spp., Neisseria spp., Propionibacterium granulosum, Stenotrophomonas maltophilia, and a combination thereof.

51. A method for classification of a gut-associated disease or cancer in a subject comprising:
(a) providing a cell-free nucleic acid sample obtained from plasma of a subject;
(b) sequencing said cell-free nucleic acid sample to provide a plurality of sequence reads;
(c) mapping said plurality of sequence reads to a human reference nucleic acid sequence;
(d) separating sequence reads that do not map to said human reference nucleic acid sequence, thereby providing presumed microbiome sequence reads;
(e) comparing said presumed microbiome sequence reads to a reference microbiome nucleic acid sequence, wherein said presumed microbiome sequence reads that map to said reference microbiome nucleic acid sequence are actual microbiome sequence reads; and
(f) using said actual microbiome sequence reads to classify said gut-associated disease or cancer in said subject.

52. The method of claim 51, wherein step (f) comprises generating a feature matrix from said actual microbiome sequence reads.

53. The method of claim 51, wherein said actual microbiome sequence reads are used to determine a relative abundance of microbiota.

54. The method of claim 51, further comprising applying principal component analysis and unsupervised machine learning to process said actual microbiome sequence reads to classify said gut-associated disease or cancer in said subject.

55. The method of claim 51, wherein said actual microbiome sequence reads represent a plurality of species of microbiota selected from Propionibacterium acnes, Candidatus Zonderia insecticola, Dasheen mosaic virus, Vicia cryptic virus, Comamonas spp., Caulobacter spp., Acinetobacter spp., Burkholdreia spp., Micrococcus luteus, Candidatus Sulcia muelleri, Torque teno virus, Polaromonas spp., Pseudomonas spp., Acinetobacter johnsonii, Cupriavidus spp., Dietzia spp., Neisseria spp., Propionibacterium granulosum, Stenotrophomonas maltophilia, and a combination thereof.

56. The method of claim 51, wherein said gut-associated disease or cancer is selected from adenoma (adenomatous polyps), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas.

57. The method of claim 51, wherein said gut-associated disease or cancer is advanced adenoma.

58. The method of claim 51, wherein said actual microbiome sequence reads identify said gut-associated disease or cancer in said subject at a sensitivity of 40% or greater and a specificity of 70% or greater.

59. The method of claim 51, wherein said actual microbiome sequence reads identify said gut-associated disease or cancer in said subject at a sensitivity of 50% or greater and a specificity of 80% or greater.

60. A method for identifying a community of microbiota in a subject comprising:
(a) providing a cell-free nucleic acid sample obtained from plasma of said subject;
(b) sequencing said cell-free nucleic acid sample to provide a plurality of sequence reads;
(c) mapping said plurality of sequence reads to a human reference nucleic acid sequence;
(d) separating sequence reads that do not map to said human reference nucleic acid sequence, thereby providing presumed microbiome sequence reads;
(e) comparing said presumed microbiome sequence reads to a reference microbiome nucleic acid sequence, wherein presumed microbiome sequence reads that map to said reference microbiome nucleic acid sequence are actual microbiome sequence reads; and
(f) using said actual microbiome sequence reads to identify said community of microbiota in said subject.

61. The method of claim 60, wherein step (f) comprises generating a feature matrix from said actual microbiome sequence reads.

62. The method of claim 60, wherein said actual microbiome sequence reads are used to determine a relative abundance of microbiota.

63. The method of claim 60, further comprising applying principal component analysis and unsupervised machine learning to process said actual microbiome sequence reads to identify said community of microbiota in said subject.

64. The method of claim 60, wherein said actual microbiome sequence reads represent a plurality of species of microbiota selected from Propionibacterium acnes, Candidatus Zonderia insecticola, Dasheen mosaic virus, Vicia cryptic virus, Comamonas spp., Caulobacter spp., Acinetobacter spp., Burkholdreia spp., Micrococcus luteus, Candidatus Sulcia muelleri, Torque teno virus, Polaromonas spp., Pseudomonas spp., Acinetobacter johnsonii, Cupriavidus spp., Dietzia spp., Neisseria spp., Propionibacterium granulosum, Stenotrophomonas maltophilia, and a combination thereof.

65. The method of claim 60, wherein said community of microbiota identified in said subject is indicative of a gut-associated disease or cancer in said subject.
